(19) United States
(12) Patent Application Publication
(10) Pub. No.: US 2025/0002853 A1
(43) Pub. Date: Jan. 2, 2025

(54) Enhanced Mobility of Inducible Pluripotent Stem Cell Derived T Regulatory Cells

(71) Applicant: CREATIVE MEDICAL TECHNOLOGIES, INC., Phoenix, AZ (US)

(72) Inventors: Thomas Ichim, San Diego, CA (US); Courney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 18/757,374

(22) Filed: Jun. 27, 2024

Related U.S. Application Data
(60) Provisional application No. 63/510,877, filed on Jun. 28, 2023.

(51) Int. Cl. C12N 5/0783

(52) U.S. Cl. CPC C12N 5/0637; C12N 2501/73; C12N 2506/1369; C12N 2510/00

(57) ABSTRACT

Cellular modifications useful for enhancing migratory potential of T regulatory cells that have been derived from inducible pluripotent stem cells. In one embodiment said inducible pluripotent stem cells are gene edited for insertion or deletion of cytoskeletal and motility associated genes in order to increase migration ability as well as deformability of cells in a manner allowing for increased extravasation and mobility. In other embodiments inducible pluripotent stem cell derived T regulatory cells are gene edited at the level of T Regulatory cell progenitor stage.

### CROSS-REFERENCE TO RELATED APPLICATIONS

The present application claims benefit of U.S. Non-Provisional Patent Application Ser. No. 63/510,877, filed on Jun. 28, 2023, entitled ENHANCED MOBILITY OF INDUCIBLE PLURIPOTENT STEM CELL DERIVED T REGULATORY CELLS, the contents of which are incorporated herein by reference in its entirety.

### FIELD OF THE INVENTION

The teachings herein relate to methods of enhancing therapeutic activity of T regulatory cells comprising enhancing mobility of said cells towards a chemotactic gradient.

### BACKGROUND OF THE INVENTION

Immunological dogma states that many autoimmune and inflammatory diseases involve autoreactive T-cells. For example, Multiple Sclerosis (MS), Rheumatoid Arthritis (RA), Systemic Lupus Erythromatosus (SLE), and Type 1 Diabetes are all autoimmune conditions. Current treatments for autoimmune and inflammatory diseases generally suppress the immune system. For example, one treatment includes transplantation of bone marrow along with administration of cytostatics and immunosuppressive drugs. Autologous hematopoietic stem cell transplantation can have lasting beneficial effects for some patients, but the procedure requires aggressive myelo-ablative conditioning which is associated with substantial toxicity and risk. Although several disease-modifying treatments (DMTs) have been approved to reduce the frequency of clinical relapses, most patients continue to clinically deteriorate under current therapy schedules. Neither DMTs nor stem cell transplantation can mediate specific suppression of the immunopathology of autoimmune and inflammatory diseases. Currently, effective treatments for autoimmune and inflammatory diseases do not exist. Treatment is focused on merely reducing its symptoms, usually by general suppression of the immune system. There is a need for a therapy which specifically targets local immune responses associated with onset and progression of disease.

Although T regulatory cell therapies have demonstrated clinical signals, these therapies need optimization.

### SUMMARY OF THE INVENTION

Preferred embodiments include methods of enhancing therapeutic activity of a T regulatory cells comprising enhancing mobility of said cell towards a chemotactic gradient.

Preferred methods include embodiments wherein said T regulatory cell is derived from a pluripotent stem cell.

Preferred methods include embodiments wherein said pluripotent stem cell is an inducible pluripotent stem cell.

Preferred methods include embodiments wherein said inducible pluripotent stem cell is generated through transfection of dedifferentiation factors into a somatic cell.

Preferred methods include embodiments wherein said somatic cell is a mesenchymal stem cell.

Preferred methods include embodiments wherein said mesenchymal stem cell is a plastic adherent cell.

Preferred methods include embodiments wherein said mesenchymal stem cell is derived from peripheral blood.

Preferred methods include embodiments wherein said mesenchymal stem cell is derived from cord blood.

Preferred methods include embodiments wherein said mesenchymal stem cell is tissue derived.

Preferred methods include embodiments wherein said tissue derived mesenchymal stem cell is isolated from Wharton's Jelly.

Preferred methods include embodiments wherein said tissue derived mesenchymal stem cell is isolated from placenta.

Preferred methods include embodiments wherein said tissue derived mesenchymal stem cell is isolated from bone marrow.

Preferred methods include embodiments wherein said tissue derived mesenchymal stem cell is isolated from umbilical cord tissue.

Preferred methods include embodiments wherein said tissue derived mesenchymal stem cell is isolated from adipose tissue.

Preferred methods include embodiments wherein said dedifferentiation factors are selected from a group comprising of: a) NANOG; b) SOX-2; c) KLF4; and d) c-myc.

Preferred methods include embodiments wherein said T regulatory cell possesses enhanced cytoskeleton mobility properties as compared to a nonmanipulated T regulatory cell.

Preferred methods include embodiments wherein said cytoskeleton is modified by enhancing expression of vimentin in T regulatory cells.

Preferred methods include embodiments wherein said expression of vimentin is enhanced by positioning the vimentin gene under control of the FoxP3 promoter.

Preferred methods include embodiments wherein said vimentin gene is additionally placed under control of HIF-1 alpha inducible promoter.

Preferred methods include embodiments wherein said therapeutic activity of said T regulatory cell is stimulation of angiogenesis.

Preferred methods include embodiments wherein said T regulatory cell induced angiogenesis is associated with augmentation of tissue matrix metalloprotease activity.

Preferred methods include embodiments wherein said matrix metalloprotease activity is mediated by MMP-1.

Preferred methods include embodiments wherein said matrix metalloprotease activity is mediated by MMP-3.

Preferred methods include embodiments wherein said matrix metalloprotease activity is mediated by MMP-5.

Preferred methods include embodiments wherein said matrix metalloprotease activity is mediated by MMP-7.

Preferred methods include embodiments wherein said matrix metalloprotease activity is mediated by MMP-9.

Preferred methods include embodiments wherein said matrix metalloprotease activity is mediated by elastase.

Preferred methods include embodiments wherein said matrix metalloprotease activity is mediated by calpain-1.

Preferred methods include embodiments wherein said matrix metalloprotease activity is mediated by calpain-2.

Preferred methods include embodiments wherein said matrix metalloprotease activity is mediated by myeloperoxidase.

Preferred methods include embodiments wherein said matrix metalloprotease activity is suppressed by TIMP-1.

Preferred methods include embodiments wherein said matrix metalloprotease activity is suppressed by TIMP-2.

Preferred methods include embodiments wherein said matrix metalloprotease activity is suppressed by TIMP-3.

Preferred methods include embodiments wherein said matrix metalloprotease activity is suppressed by TIMP-4.

Preferred methods include embodiments wherein said T regulatory cell induced angiogenesis is associated with endothelial cell migration towards a hypoxic gradient.

Preferred methods include embodiments wherein said T regulatory cell induced angiogenesis is associated with monocyte migration towards a hypoxic gradient.

Preferred methods include embodiments wherein said T regulatory cell induced angiogenesis is associated with fibroblast migration towards a hypoxic gradient.

Preferred methods include embodiments wherein said T regulatory cell induced angiogenesis is associated with mast cell migration towards a hypoxic gradient.

Preferred methods include embodiments wherein said T regulatory cell induced angiogenesis is associated with increased interleukin-1 beta receptor antagonist in the area of new blood vessel formation.

Preferred methods include embodiments wherein said T regulatory cell induced angiogenesis is associated with increased interleukin-3 in the area of new blood vessel formation.

Preferred methods include embodiments wherein said T regulatory cell induced angiogenesis is associated with increased interleukin-5 in the area of new blood vessel formation.

Preferred methods include embodiments wherein said T regulatory cell induced angiogenesis is associated with increased interleukin-7 in the area of new blood vessel formation.

Preferred methods include embodiments wherein said T regulatory cell induced angiogenesis is associated with increased interleukin-8 in the area of new blood vessel formation.

Preferred methods include embodiments wherein said T regulatory cell induced angiogenesis is associated with increased interleukin-10 in the area of new blood vessel formation.

Preferred methods include embodiments wherein said T regulatory cell induced angiogenesis is associated with increased interleukin-13 in the area of new blood vessel formation.

Preferred methods include embodiments wherein said T regulatory cell induced angiogenesis is associated with increased interleukin-15 in the area of new blood vessel formation.

Preferred methods include embodiments wherein said T regulatory cell induced angiogenesis is associated with increased interleukin-17 in the area of new blood vessel formation.

Preferred methods include embodiments wherein said T regulatory cell induced angiogenesis is associated with increased interleukin-22 in the area of new blood vessel formation.

Preferred methods include embodiments wherein said T regulatory cell induced angiogenesis is associated with increased interleukin-25 in the area of new blood vessel formation.

Preferred methods include embodiments wherein said T regulatory cell induced angiogenesis is associated with increased interleukin-35 in the area of new blood vessel formation.

Preferred methods include embodiments wherein said T regulatory cell induced angiogenesis is associated with increased interleukin-37 in the area of new blood vessel formation.

Preferred methods include embodiments wherein said T regulatory cell induced angiogenesis is associated with increased interleukin-38 in the area of new blood vessel formation.

Preferred methods include embodiments wherein said T regulatory cell induced angiogenesis is associated with increased FGF-1 in the area of new blood vessel formation.

Preferred methods include embodiments wherein said T regulatory cell induced angiogenesis is associated with increased FGF-2 in the area of new blood vessel formation.

Preferred methods include embodiments wherein said T regulatory cell induced angiogenesis is associated with increased FGF-5 in the area of new blood vessel formation.

Preferred methods include embodiments wherein said T regulatory cell induced angiogenesis is associated with increased FGF-7 in the area of new blood vessel formation.

Preferred methods include embodiments wherein said T regulatory cell induced angiogenesis is associated with increased FGF-10 in the area of new blood vessel formation.

Preferred methods include embodiments wherein said T regulatory cell induced angiogenesis is associated with increased FGF-17 in the area of new blood vessel formation.

Preferred methods include embodiments wherein said T regulatory cell induced angiogenesis is associated with increased FGF-18 in the area of new blood vessel formation.

Preferred methods include embodiments wherein said T regulatory cell induced angiogenesis is associated with increased FGF-21 in the area of new blood vessel formation.

Preferred methods include embodiments wherein said T regulatory cell induced angiogenesis is associated with increased FGF-22 in the area of new blood vessel formation.

Preferred methods include embodiments wherein said T regulatory cell induced angiogenesis is associated with increased FGF-23 in the area of new blood vessel formation.

Preferred methods include embodiments wherein said T regulatory cell induced angiogenesis is associated with increased EGF in the area of new blood vessel formation.

Preferred methods include embodiments wherein said T regulatory cell induced angiogenesis is associated with increased VEGF in the area of new blood vessel formation.

Preferred methods include embodiments wherein said T regulatory cell induced angiogenesis is associated with increased VEGF-C in the area of new blood vessel formation.

Preferred methods include embodiments wherein said T regulatory cell induced angiogenesis is associated with increased CNTF in the area of new blood vessel formation.

Preferred methods include embodiments wherein said T regulatory cell induced angiogenesis is associated with increased angiopoietin in the area of new blood vessel formation.

Preferred methods include embodiments wherein said T regulatory cell induced angiogenesis is associated with increased osteopontin in the area of new blood vessel formation.

Preferred methods include embodiments wherein said T regulatory cell induced angiogenesis is associated with increased GDF-11 in the area of new blood vessel formation.

Preferred methods include embodiments wherein said T regulatory cell induced angiogenesis is associated with increased GDF-15 in the area of new blood vessel formation.

Preferred methods include embodiments wherein said T regulatory cell induced angiogenesis is associated with increased Endoglin in the area of new blood vessel formation.

Preferred methods include embodiments wherein said T regulatory cell induced angiogenesis is associated with increased soluble TNF alpha receptor p55 in the area of new blood vessel formation.

Preferred methods include embodiments wherein said T regulatory cell induced angiogenesis is associated with increased soluble TNF alpha receptor p75 in the area of new blood vessel formation.

Preferred methods include embodiments wherein said T regulatory cell induced angiogenesis is associated with increased PDGF-BB in the area of new blood vessel formation.

Preferred methods include embodiments wherein said therapeutic activity of said T regulatory cell is stimulation of endogenous progenitor cells.

Preferred methods include embodiments wherein said endogenous progenitor cells are cardiac stem specific stem cells.

Preferred methods include embodiments wherein said cardiac specific stem cells express c-kit.

Preferred methods include embodiments wherein said cardiac specific stem cells express Notch.

Preferred methods include embodiments wherein said cardiac specific stem cells express surface vimentin.

Preferred methods include embodiments wherein said cardiac specific stem cells express CD117.

Preferred methods include embodiments wherein said cardiac specific stem cells express OCT4.

Preferred methods include embodiments wherein said cardiac specific stem cells express CD127.

Preferred methods include embodiments wherein said cardiac specific stem cells express PD-L1.

Preferred methods include embodiments wherein said cardiac specific stem cells express TGF-beta receptor.

Preferred methods include embodiments wherein said cardiac specific stem cells express aldehyde dehydrogenase.

Preferred methods include embodiments wherein said cardiac specific stem cells express CD83.

Preferred methods include embodiments wherein said cardiac specific stem cells express CD133.

Preferred methods include embodiments wherein said cardiac specific stem cells express osteopontin receptor.

Preferred methods include embodiments wherein said cardiac specific stem cells express RANK.

Preferred methods include embodiments wherein said cardiac specific stem cells express jagged.

Preferred methods include embodiments wherein said cardiac specific stem cells express angiopoietin receptor.

Preferred methods include embodiments wherein said cardiac specific stem cells express c-met.

Preferred methods include embodiments wherein said cardiac specific stem cells express C-IAP.

Preferred methods include embodiments wherein said cardiac specific stem cells express NANOG.

Preferred methods include embodiments wherein said cardiac specific stem cells express myosin.

Preferred methods include embodiments wherein said cardiac specific stem cells express tubulin.

Preferred methods include embodiments wherein said cardiac specific stem cells express ABC efflux pump.

Preferred methods include embodiments wherein said cardiac specific stem cells possess ability to undergo asymmetric division.

Preferred methods include embodiments wherein said cardiac specific stem cells can differentiate into cardiomyocytes in the presence of a tissue damage associated signal.

Preferred methods include embodiments wherein said tissue damage associated signal is an activator of one or more toll like receptors.

Preferred methods include embodiments wherein said endogenous progenitor cells are neurogenic stem cells.

Preferred methods include embodiments wherein said neurogenic stem cells divide asymmetrically when treated with T regulatory cells.

Preferred methods include embodiments wherein said neurogenic stem cells divide asymmetrically when treated with T regulatory cells and HGF-1.

Preferred methods include embodiments wherein said neurogenic stem cells divide asymmetrically when treated with T regulatory cells and human chorionic gonadotropin.

Preferred methods include embodiments wherein said neurogenic stem cells divide asymmetrically when treated with T regulatory cells and M-CSF.

Preferred methods include embodiments wherein said neurogenic stem cells divide asymmetrically when treated with T regulatory cells and G-CSF.

Preferred methods include embodiments wherein said neurogenic stem cells divide asymmetrically when treated with T regulatory cells and GM-CSF.

Preferred methods include embodiments wherein said neurogenic stem cells divide asymmetrically when treated with T regulatory cells and interleukin-22.

Preferred methods include embodiments wherein said neurogenic stem cells divide asymmetrically when treated with T regulatory cells and FGF-1.

Preferred methods include embodiments wherein said neurogenic stem cells divide asymmetrically when treated with T regulatory cells and FGF-2.

Preferred methods include embodiments wherein said neurogenic stem cells divide asymmetrically when treated with T regulatory cells and type 2 monocytes.

Preferred methods include embodiments wherein said neurogenic stem cells divide asymmetrically when treated with T regulatory cells and type 2 astrocytes.

Preferred methods include embodiments wherein said neurogenic stem cells divide asymmetrically when treated with T regulatory cells and type 2 neutrophils.

Preferred methods include embodiments wherein said neurogenic stem cells divide asymmetrically when treated with T regulatory cells and monocytes engineered to express interleukin-10.

Preferred methods include embodiments wherein said neurogenic stem cells divide asymmetrically when treated with T regulatory cells and monocytes engineered to express NGF.

Preferred methods include embodiments wherein said neurogenic stem cells divide asymmetrically when treated with T regulatory cells and monocytes engineered to express BDNF.

Preferred methods include embodiments wherein said neurogenic stem cells divide asymmetrically when treated with T regulatory cells and monocytes engineered to express CNTF.

Preferred methods include embodiments wherein said neurogenic stem cells divide asymmetrically when treated with T regulatory cells and monocytes engineered to express NOTCH.

Preferred methods include embodiments wherein said neurogenic stem cells divide asymmetrically when treated with T regulatory cells and monocytes engineered to express angiopoietin.

Preferred methods include embodiments wherein said neurogenic stem cells divide asymmetrically when treated with T regulatory cells and monocytes engineered to express GC-MAF.

Preferred methods include embodiments wherein said neurogenic stem cells reside in the dentate gyrus.

Preferred methods include embodiments wherein said neurogenic stem cells reside in the subventricular zone

Preferred methods include embodiments wherein said neurogenic stem cells are capable of decreasing penumbra size after a cerebral infarct.

Preferred methods include embodiments wherein said neurogenic stem cells are capable of increase neural plasticity after a cerebral infarct.

Preferred methods include embodiments wherein said neurogenic stem cells are capable of restoring cognitive function subsequent to an increase in neuroinflammatory cytokines.

Preferred methods include embodiments wherein said neuroinflammatory cytokine is HMGB-1.

Preferred methods include embodiments wherein said neuroinflammatory cytokine is soluble neuropilin.

Preferred methods include embodiments wherein said neuroinflammatory cytokine is extracellular histones.

Preferred methods include embodiments wherein said neuroinflammatory cytokine is extracellular calreticulin.

Preferred methods include embodiments wherein said neuroinflammatory cytokine is TNF-alpha.

Preferred methods include embodiments wherein said neuroinflammatory cytokine is lymphotoxin.

Preferred methods include embodiments wherein said neuroinflammatory cytokine is activated nitric oxide synthase.

Preferred methods include embodiments wherein said neuroinflammatory cytokine is collage I fragments.

Preferred methods include embodiments wherein said neuroinflammatory cytokine is collage II fragments.

Preferred methods include embodiments wherein said neuroinflammatory cytokine is low molecular weight hyaluronic acid.

Preferred methods include embodiments wherein said neuroinflammatory cytokine is interleukin beta.

Preferred methods include embodiments wherein said neuroinflammatory cytokine is interleukin 6.

Preferred methods include embodiments wherein said neuroinflammatory cytokine is interleukin 8.

Preferred methods include embodiments wherein said neuroinflammatory cytokine is interleukin 9.

Preferred methods include embodiments wherein said neuroinflammatory cytokine is interleukin1.

Preferred methods include embodiments wherein said neuroinflammatory cytokine is interleukin2.

Preferred methods include embodiments wherein said neuroinflammatory cytokine is interleukin5.

Preferred methods include embodiments wherein said neuroinflammatory cytokine is interleukin7.

Preferred methods include embodiments wherein said neuroinflammatory cytokine is interleukin8.

Preferred methods include embodiments wherein said neuroinflammatory cytokine is interleukin 21.

Preferred methods include embodiments wherein said neuroinflammatory cytokine is interleukin 22.

Preferred methods include embodiments wherein said neuroinflammatory cytokine is interleukin 23.

Preferred methods include embodiments wherein said neuroinflammatory cytokine is interleukin 27.

Preferred methods include embodiments wherein said neuroinflammatory cytokine is interleukin 33.

Preferred methods include embodiments wherein said neuroinflammatory cytokine is MIP-1 alpha.

Preferred methods include embodiments wherein said neuroinflammatory cytokine is MIP-1 beta.

Preferred methods include embodiments wherein said neuroinflammatory cytokine is MCP-1.

Preferred methods include embodiments wherein said endogenous progenitor cells are osteoblastic cells.

Preferred methods include embodiments wherein said osteoblastic cells are capable of generating bone cells.

Preferred methods include embodiments wherein said osteoblastic cells are inhibited by RANK ligand.

Preferred methods include embodiments wherein said endogenous progenitor cells are hepatic progenitor cells.

Preferred methods include embodiments wherein said hepatic progenitor cells are capable of suppressing activity of stellate cells.

Preferred methods include embodiments wherein said hepatic progenitor cells are capable of efflux of rhodamine 231.

Preferred methods include embodiments wherein said hepatic progenitor cells express CD105.

Preferred methods include embodiments wherein said hepatic progenitor cells express CD90.

Preferred methods include embodiments wherein said hepatic progenitor cells express KLF4.

Preferred methods include embodiments wherein said hepatic progenitor cells express PDX-1.

Preferred methods include embodiments wherein said hepatic progenitor cells express c-met.

Preferred methods include embodiments wherein said hepatic progenitor cells express PDGF-BB receptor.

Preferred methods include embodiments wherein said hepatic progenitor cells express EGF receptor.

Preferred methods include embodiments wherein said hepatic progenitor cells express IGF-1 receptor.

Preferred methods include embodiments wherein said hepatic progenitor cells express livin.

Preferred methods include embodiments wherein said hepatic progenitor cells express survivin.

Preferred methods include embodiments wherein said hepatic progenitor cells express bcl-2.

Preferred methods include embodiments wherein said hepatic progenitor cells express bcl-2XL.

Preferred methods include embodiments wherein said hepatic progenitor cells express scramblase.

Preferred methods include embodiments wherein said hepatic progenitor cells express c-met.

Preferred methods include embodiments wherein said endogenous progenitor cells are nucleus pulposus cells.

Preferred methods include embodiments wherein said endogenous progenitor cells are dermal progenitor cells.

Preferred methods include embodiments wherein said endogenous progenitor cells are chondrocyte progenitor cells.

Preferred methods include embodiments wherein said endogenous progenitor cells are nephrogenic progenitor cells.

Preferred methods include embodiments wherein said endogenous progenitor cells are type 2 pulmonary epithelial cells.

Preferred methods include embodiments wherein said endogenous progenitor cells are colonic epithelial cell progenitors.

Preferred methods include embodiments wherein said endogenous progenitor cells are gastric epithelial cell progenitors.

Preferred methods include embodiments wherein said endogenous progenitor cells are pancreatic progenitors.

Preferred methods include embodiments wherein said therapeutic activity of said T regulatory cell is suppression of apoptosis.

81, Preferred methods include embodiments wherein said suppression of apoptosis occurs in tissue affected by a disease process.

Preferred methods include embodiments wherein said disease process is an inflammatory disease process.

Preferred methods include embodiments wherein said disease process is an autoimmune disease process.

Preferred methods include embodiments wherein said disease process is a genetic based disease process.

81, Preferred methods include embodiments wherein said suppression of apoptosis is associated with augmented antioxidant enzymes inside said cell at risk of apoptosis.

Preferred methods include embodiments wherein said antioxidant enzyme is catalase.

Preferred methods include embodiments wherein said antioxidant enzyme is a superoxide dismutase.

Preferred methods include embodiments wherein said superoxide dismutase is manganese dependent.

Preferred methods include embodiments wherein said superoxide dismutase is zinc dependent.

Preferred methods include embodiments wherein said antioxidant enzyme is xanthine oxidase.

Preferred methods include embodiments wherein said antioxidant enzyme is glutathione.

Preferred methods include embodiments wherein said autoimmune disease is mediated by T cells.

Preferred methods include embodiments wherein said T cell is a Th1 cell.

Preferred methods include embodiments wherein said T cell is a Th9 cell.

Preferred methods include embodiments wherein said T cell is a Th17 cell.

Preferred methods include embodiments wherein said Th1 cell expresses STAT3.

Preferred methods include embodiments wherein said Th1 cell secretes more interferon gamma as compared to interleukin-4 when activated.

Preferred methods include embodiments wherein said T cell activation is crosslinking of the T cell receptor in an antigen specific manner.

Preferred methods include embodiments wherein said T cell activation is crosslinking of the T cell receptor in an antigen non-specific manner.

Preferred methods include embodiments wherein said T cell activation is accomplished by treatment with a calcium ionophore.

Preferred methods include embodiments wherein said T cell activation is accomplished by treatment with a mitogen.

Preferred methods include embodiments wherein said mitogen is concanavalin A.

Preferred methods include embodiments wherein said mitogen is pokeweed mitogen.

Preferred methods include embodiments wherein said mitogen is cyaninvirin.

Preferred methods include embodiments wherein said mitogen is phytohemagglutinin.

Preferred methods include embodiments wherein said mitogen is a solid surface containing anti-CD3 and anti-CD28.

Preferred methods include embodiments wherein said enhanced migration of said T regulatory cell is provided for by treatment of said T regulatory cell with a dedifferentiation agent or a combination of a dedifferentiation agent and a chemotaxis promoting agent.

Preferred methods include embodiments wherein said dedifferentiation agent is a histone deacetylase inhibitor.

Preferred methods include embodiments wherein said histone deacetylase inhibitor is valproic acid.

Preferred methods include embodiments wherein said histone deacetylase inhibitor is trichostatin A.

Preferred methods include embodiments wherein said histone deacetylase inhibitor is phenylbutyrate.

Preferred methods include embodiments wherein said histone deacetylase inhibitor is sodium phenylbutyrate.

Preferred methods include embodiments wherein said histone deacetylase inhibitor is sulforaphane.

Preferred methods include embodiments wherein said histone deacetylase inhibitor is one or more short interfering RNA molecules blocking expression of histone deacetylases.

Preferred methods include embodiments wherein said histone deacetylase inhibitor is one or more antisense oligonucleotide molecules blocking expression of histone deacetylases.

Preferred methods include embodiments wherein said histone deacetylase inhibitor is one or more decoy oligonucleotides capable of blocking interaction between histone deacetylases and DNA.

Preferred methods include embodiments wherein said dedifferentiation agent is cytoplasmic extract from a pluripotent stem cell.

Preferred methods include embodiments wherein said pluripotent stem cell is an embryonic stem cell.

Preferred methods include embodiments wherein said pluripotent stem cell is a pluripotent stem cell.

Preferred methods include embodiments wherein said pluripotent stem cell is an inducible pluripotent stem cell.

Preferred methods include embodiments wherein said pluripotent stem cell is stress induced pluripotent stem cell.

Preferred methods include embodiments wherein said pluripotent stem cell is a parthenogenesis induced pluripotent stem cell.

Preferred methods include embodiments wherein said dedifferentiation agent is mRNA extracted from a pluripotent stem cell.

Preferred methods include embodiments wherein said pluripotent stem cell is a somatic cell nuclear transplant derived stem cell.

Preferred methods include embodiments wherein said dedifferentiation agent is cytoplasmic extract from a pluripotent stem cell.

Preferred methods include embodiments wherein said pluripotent stem cell is an embryonic stem cell.

Preferred methods include embodiments wherein said pluripotent stem cell is a pluripotent stem cell.

Preferred methods include embodiments wherein said pluripotent stem cell is an inducible pluripotent stem cell.

Preferred methods include embodiments wherein said pluripotent stem cell is stress induced pluripotent stem cell.

Preferred methods include embodiments wherein said pluripotent stem cell is a parthenogenesis induced pluripotent stem cell.

Preferred methods include embodiments wherein said pluripotent stem cell is a somatic cell nuclear transplant derived stem cell.

Preferred methods include embodiments wherein said dedifferentiation agent is microRNA extracted from a pluripotent stem cell.

Preferred methods include embodiments wherein said pluripotent stem cell is a somatic cell nuclear transplant derived stem cell.

Preferred methods include embodiments wherein said dedifferentiation agent is cytoplasmic extract from a pluripotent stem cell.

Preferred methods include embodiments wherein said pluripotent stem cell is an embryonic stem cell.

Preferred methods include embodiments wherein said pluripotent stem cell is a pluripotent stem cell.

Preferred methods include embodiments wherein said pluripotent stem cell is an inducible pluripotent stem cell.

Preferred methods include embodiments wherein said pluripotent stem cell is stress induced pluripotent stem cell.

Preferred methods include embodiments wherein said pluripotent stem cell is a parthenogenesis induced pluripotent stem cell.

Preferred methods include embodiments wherein said pluripotent stem cell is a somatic cell nuclear transplant derived stem cell.

Preferred methods include embodiments wherein said dedifferentiation agent is mRNA encoding OCT4.

Preferred methods include embodiments wherein said dedifferentiation agent is mRNA encoding NANOG.

Preferred methods include embodiments wherein said dedifferentiation agent is mRNA encoding FOXP3.

Preferred methods include embodiments wherein said dedifferentiation agent is mRNA encoding AIRE.

Preferred methods include embodiments wherein said dedifferentiation agent is mRNA encoding SOX2.

Preferred methods include embodiments wherein said dedifferentiation agent is mRNA encoding RAS.

Preferred methods include embodiments wherein said dedifferentiation agent is mRNA encoding RAF.

Preferred methods include embodiments wherein said dedifferentiation agent is mRNA encoding c-myc.

Preferred methods include embodiments wherein said dedifferentiation agent is mRNA encoding PIM-1.

Preferred methods include embodiments wherein said dedifferentiation agent is mRNA encoding shRNA to vimentin.

Preferred methods include embodiments wherein said dedifferentiation agent is mRNA encoding shRNA to p53.

Preferred methods include embodiments wherein said dedifferentiation agent is mRNA encoding bcr-abl.

Preferred methods include embodiments wherein said dedifferentiation agent is mRNA encoding EGF receptor.

Preferred methods include embodiments wherein said dedifferentiation agent is mRNA encoding Brother of the Regulator of Imprinted Sites.

Preferred methods include embodiments wherein said dedifferentiation agent is mRNA encoding c-met.

Preferred methods include embodiments wherein said dedifferentiation agent is mRNA encoding SV40 large T antigen.

Preferred methods include embodiments wherein said dedifferentiation agent is mRNA encoding syndecan.

Preferred methods include embodiments wherein said dedifferentiation agent is mRNA encoding interleukin-13.

Preferred methods include embodiments wherein said dedifferentiation agent is mRNA encoding interleukin-22.

Preferred methods include embodiments wherein said dedifferentiation agent is mRNA encoding HER2.

Preferred methods include embodiments wherein said dedifferentiation agent is mRNA encoding TWIST.

Preferred methods include embodiments wherein said dedifferentiation agent is mRNA encoding the Son of Sevenless.

Preferred methods include embodiments wherein said dedifferentiation agent is mRNA encoding VEGF-C.

Preferred methods include embodiments wherein said dedifferentiation agent is mRNA encoding TGF-beta.

Preferred methods include embodiments wherein said dedifferentiation agent is mRNA encoding endoglin.

Preferred methods include embodiments wherein said dedifferentiation agent is mRNA encoding FGF-1.

Preferred methods include embodiments wherein said dedifferentiation agent is mRNA encoding FGF-2.

Preferred methods include embodiments wherein said dedifferentiation agent is mRNA encoding FGF-5.

Preferred methods include embodiments wherein said dedifferentiation agent is mRNA encoding EGF.

Preferred methods include embodiments wherein said dedifferentiation agent is pDNA encoding OCT4.

Preferred methods include embodiments wherein said dedifferentiation agent is pDNA encoding NANOG.

Preferred methods include embodiments wherein said dedifferentiation agent is pDNA encoding FOXP3.

Preferred methods include embodiments wherein said dedifferentiation agent is pDNA encoding AIRE.

Preferred methods include embodiments wherein said dedifferentiation agent is pDNA encoding SOX2.

Preferred methods include embodiments wherein said dedifferentiation agent is pDNA encoding RAS.

Preferred methods include embodiments wherein said dedifferentiation agent is pDNA encoding RAF.

Preferred methods include embodiments wherein said dedifferentiation agent is pDNA encoding c-myc.

Preferred methods include embodiments wherein said dedifferentiation agent is pDNA encoding PIM-1.

Preferred methods include embodiments wherein said dedifferentiation agent is pDNA encoding shRNA to vimentin.

Preferred methods include embodiments wherein said dedifferentiation agent is pDNA encoding shRNA to p53.

Preferred methods include embodiments wherein said dedifferentiation agent is pDNA encoding bcr-abl.

Preferred methods include embodiments wherein said dedifferentiation agent is pDNA encoding EGF receptor.

Preferred methods include embodiments wherein said dedifferentiation agent is pDNA encoding Brother of the Regulator of Imprinted Sites.

Preferred methods include embodiments wherein said dedifferentiation agent is pDNA encoding c-met.

Preferred methods include embodiments wherein said dedifferentiation agent is pDNA encoding SV40 large T antigen.

Preferred methods include embodiments wherein said dedifferentiation agent is pDNA encoding syndecan.

Preferred methods include embodiments wherein said dedifferentiation agent is pDNA encoding interleukin-13.

Preferred methods include embodiments wherein said dedifferentiation agent is pDNA encoding interleukin-22.

Preferred methods include embodiments wherein said dedifferentiation agent is pDNA encoding HER2.

Preferred methods include embodiments wherein said dedifferentiation agent is pDNA encoding TWIST.

Preferred methods include embodiments wherein said dedifferentiation agent is pDNA encoding the Son of Sevenless.

Preferred methods include embodiments wherein said dedifferentiation agent is pDNA encoding VEGF-C.

Preferred methods include embodiments wherein said dedifferentiation agent is pDNA encoding TGF-beta.

Preferred methods include embodiments wherein said dedifferentiation agent is pDNA encoding endoglin.

Preferred methods include embodiments wherein said dedifferentiation agent is pDNA encoding FGF-1.

Preferred methods include embodiments wherein said dedifferentiation agent is pDNA encoding FGF-2.

Preferred methods include embodiments wherein said dedifferentiation agent is pDNA encoding FGF-5.

Preferred methods include embodiments wherein said dedifferentiation agent is pDNA encoding EGF.

Preferred methods include embodiments wherein said cell is transfected with a T regulatory cell activity enhancing gene.

Preferred methods include embodiments wherein said T regulatory cell activity enhancing gene is ACAA2.

Preferred methods include embodiments wherein said T regulatory cell activity enhancing gene is ACADM.

Preferred methods include embodiments wherein said T regulatory cell activity enhancing gene is ACADVL.

Preferred methods include embodiments wherein said T regulatory cell activity enhancing gene is ACOT7.

Preferred methods include embodiments wherein said T regulatory cell activity enhancing gene is ACSL1.

Preferred methods include embodiments wherein said T regulatory cell activity enhancing gene is ACSL4.

Preferred methods include embodiments wherein said T regulatory cell activity enhancing gene is ACSL5.

Preferred methods include embodiments wherein said T regulatory cell activity enhancing gene is AGK.

Preferred methods include embodiments wherein said T regulatory cell activity enhancing gene is AGMAT.

Preferred methods include embodiments wherein said T regulatory cell activity enhancing gene is AK4.

Preferred methods include embodiments wherein said T regulatory cell activity enhancing gene is ARG2.

Preferred methods include embodiments wherein said T regulatory cell activity enhancing gene is ARL2.

Preferred methods include embodiments wherein said T regulatory cell activity enhancing gene is AUH.

Preferred methods include embodiments wherein said T regulatory cell activity enhancing gene is BCL2L1.

Preferred methods include embodiments wherein said T regulatory cell activity enhancing gene is BDH1.

Preferred methods include embodiments wherein said T regulatory cell activity enhancing gene is BNIP1.

Preferred methods include embodiments wherein said T regulatory cell activity enhancing gene is CDK1.

Preferred methods include embodiments wherein said T regulatory cell activity enhancing gene is CHDH.

Preferred methods include embodiments wherein said T regulatory cell activity enhancing gene is CIAPIN1.

Preferred methods include embodiments wherein said T regulatory cell activity enhancing gene is CISD2.

Preferred methods include embodiments wherein said T regulatory cell activity enhancing gene is COX17.

Preferred methods include embodiments wherein said T regulatory cell activity enhancing gene is CPT1A.

Preferred methods include embodiments wherein said T regulatory cell activity enhancing gene is CPT2.

Preferred methods include embodiments wherein said T regulatory cell activity enhancing gene is CYB5B.

Preferred methods include embodiments wherein said T regulatory cell activity enhancing gene is DAP3.

Preferred methods include embodiments wherein said T regulatory cell activity enhancing gene is DHRS2.

Preferred methods include embodiments wherein said T regulatory cell activity enhancing gene is DNM1L.

Preferred methods include embodiments wherein said T regulatory cell activity enhancing gene is DUT.

Preferred methods include embodiments wherein said T regulatory cell activity enhancing gene is DYNLL1.

Preferred methods include embodiments wherein said T regulatory cell activity enhancing gene is ECI1.

Preferred methods include embodiments wherein said T regulatory cell activity enhancing gene is FDXR.

Preferred methods include embodiments wherein said T regulatory cell activity enhancing gene is FEN1.

Preferred methods include embodiments wherein said T regulatory cell activity enhancing gene is FKBP8.

Preferred methods include embodiments wherein said T regulatory cell activity enhancing gene is GK.

Preferred methods include embodiments wherein said T reg9latory cell activity enhancing gene is GRSF1.

Preferred methods include embodiments wherein said T regulatory cell activity enhancing gene is HTRA2.

Preferred methods include embodiments wherein said T regulatory cell activity enhancing gene is L2HGDH.

Preferred methods include embodiments wherein said T regulatory cell activity enhancing gene is LRPPRC.

Preferred methods include embodiments wherein said T regulatory cell activity enhancing gene is MAIP1.

Preferred methods include embodiments wherein said T regulatory cell activity enhancing gene is MAOA.

Preferred methods include embodiments wherein said T regulatory cell activity enhancing gene is MPST.

Preferred methods include embodiments wherein said T regulatory cell activity enhancing gene is MRPL1.

Preferred methods include embodiments wherein said T regulatory cell activity enhancing gene is MRPL13.

Preferred methods include embodiments wherein said T regulatory cell activity enhancing gene is MRPL14.

Preferred methods include embodiments wherein said T regulatory cell activity enhancing gene is MRPL17.

Preferred methods include embodiments wherein said T regulatory cell activity enhancing gene is MRPL22.

Preferred methods include embodiments wherein said T regulatory cell activity enhancing gene is MRPL37.

Preferred methods include embodiments wherein said T regulatory cell activity enhancing gene is MRPL39.

Preferred methods include embodiments wherein said T regulatory cell activity enhancing gene is MRPL4.

Preferred methods include embodiments wherein said T regulatory cell activity enhancing gene is MRPL43.

Preferred methods include embodiments wherein said T regulatory cell activity enhancing gene is MRPL44.

Preferred methods include embodiments wherein said T regulatory cell activity enhancing gene is MRPL46.

Preferred methods include embodiments wherein said T regulatory cell activity enhancing gene is MRPL48.

Preferred methods include embodiments wherein said T regulatory cell activity enhancing gene is MRPS11.

Preferred methods include embodiments wherein said T regulatory cell activity enhancing gene is MRPS14.

Preferred methods include embodiments wherein said T regulatory cell activity enhancing gene is MRPS2.

Preferred methods include embodiments wherein said T regulatory cell activity enhancing gene is MRPS27.

Preferred methods include embodiments wherein said T regulatory cell activity enhancing gene is MRPS31.

Preferred methods include embodiments wherein said T regulatory cell activity enhancing gene is MRPS35.

Preferred methods include embodiments wherein said T regulatory cell activity enhancing gene is MTHFD2.

Preferred methods include embodiments wherein said T regulatory cell activity enhancing gene is MTX1.

Preferred methods include embodiments wherein said T regulatory cell activity enhancing gene is MYCBP.

Preferred methods include embodiments wherein said T regulatory cell activity enhancing gene is NDUFA8.

Preferred methods include embodiments wherein said T regulatory cell activity enhancing gene is NUDT1.

Preferred methods include embodiments wherein said T regulatory cell activity enhancing gene is OAT.

Preferred methods include embodiments wherein said T regulatory cell activity enhancing gene is PITRM1.

Preferred methods include embodiments wherein said T regulatory cell activity enhancing gene is PLSCR3.

Preferred methods include embodiments wherein said T regulatory cell activity enhancing gene is PMPCA.

Preferred methods include embodiments wherein said T regulatory cell activity enhancing gene is PPIF.

Preferred methods include embodiments wherein said T regulatory cell activity enhancing gene is PRH2.

Preferred methods include embodiments wherein said T regulatory cell activity enhancing gene is PYCR2.

Preferred methods include embodiments wherein said T regulatory cell activity enhancing gene is REXO2.

Preferred methods include embodiments wherein said T regulatory cell activity enhancing gene is RMND1.

Preferred methods include embodiments wherein said T regulatory cell activity enhancing gene is SLC25A10.

Preferred methods include embodiments wherein said T regulatory cell activity enhancing gene is SLC25A19.

Preferred methods include embodiments wherein said T regulatory cell activity enhancing gene is SLC25A4.

Preferred methods include embodiments wherein said T regulatory cell activity enhancing gene is TIGAR.

Preferred methods include embodiments wherein said T regulatory cell activity enhancing gene is TIMM13.

Preferred methods include embodiments wherein said T regulatory cell activity enhancing gene is TIMM23.

Preferred methods include embodiments wherein said T regulatory cell activity enhancing gene is TMEM14C.

Preferred methods include embodiments wherein said T regulatory cell activity enhancing gene is TOMM22.

Preferred methods include embodiments wherein said T regulatory cell activity enhancing gene is TOMM34.

Preferred methods include embodiments wherein said T regulatory cell activity enhancing gene is TOMM40.

Preferred methods include embodiments wherein said T regulatory cell activity enhancing gene is TST.

Preferred methods include embodiments wherein said T regulatory cells are administered in a manner to augment numbers and/or activity of myeloid derived suppressor cells (MDSCs).

Preferred methods include embodiments wherein said MDSCs are capable of reducing proliferation of T cells stimulating with a mitogen.

Preferred methods include embodiments wherein said MDSCs are capable of reducing cytokine production of T cells stimulating with a mitogen.

Preferred methods include embodiments wherein said cytokine production being reduced occurs to cytokines of the Th1 family.

Preferred methods include embodiments wherein said Th1 family cytokine is TNF-alpha.

Preferred methods include embodiments wherein said Th1 family cytokine is lymphotoxin.

Preferred methods include embodiments wherein said Th1 family cytokine is interleukin-2.

Preferred methods include embodiments wherein said Th1 family cytokine is interleukin-12.

Preferred methods include embodiments wherein said Th1 family cytokine is interleukin-15.

Preferred methods include embodiments wherein said Th1 family cytokine is interleukin-18.

Preferred methods include embodiments wherein said Th1 family cytokine is interleukin-23.

Preferred methods include embodiments wherein said MDSCs reduce T cell activation through stimulation of the enzyme indolamine 3 dioxygenase.

Preferred methods include embodiments wherein said MDSCs reduce T cell activation through stimulation of the enzyme arginase.

Preferred methods include embodiments wherein said MDSCs reduce T cell activation through stimulation of the inducible nitric oxide synthase.

Preferred methods include embodiments wherein said MDSCs are generated by administration of a myeloid specific cellular mobilizer together with one or more populations of T regulatory cells.

Preferred methods include embodiments wherein said myeloid mobilizer is TGF-beta.

Preferred methods include embodiments wherein said myeloid mobilizer is HGF-1.

Preferred methods include embodiments wherein said myeloid mobilizer is TNF-alpha.

Preferred methods include embodiments wherein said myeloid mobilizer is G-CSF.

Preferred methods include embodiments wherein said myeloid mobilizer is M-CSF.

Preferred methods include embodiments wherein said myeloid mobilizer is GM-CSF.

Preferred methods include embodiments wherein said myeloid mobilizer is hCG.

Preferred methods include embodiments wherein said myeloid mobilizer is oxytocin.

Preferred methods include embodiments wherein said myeloid mobilizer is angiopoietin.

Preferred methods include embodiments wherein said myeloid mobilizer is mesenchymal stem cell conditioned media.

### DETAILED DESCRIPTION OF THE INVENTION

The invention teaches the enhancement of T regulatory (Treg) cell mobility and deformability through alteration of cytoskeletal proteins such as vimentin. In particular the invention provides, intra alia, pluripotent stem cell derived Treg cells which are modified to express less vimentin as compared to wild-type T regulatory cells. The decreased vimentin expression causes enhanced deformability of the Treg cell. Additionally, Treg cells possessing enhanced chemokine receptor expression and enhanced matrix metalloprotease activity are provided.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

For the practice of the invention, the term “regulatory T cell” (Treg) means a T cell which expresses the markers CD4, CD25 and FOXP3 (CD4+CD25+FOXP3+). Tregs may be identified using the cell surface markers CD4 and CD25 in the absence of or in combination with low-level expression of the surface protein CD127 (CD4+CD25+CD127− or CD4+CD25+CD127low). Tregs may also express on the cell surface high levels of CTLA-4 (cytotoxic T-lymphocyte associated molecule-4) or GITR (glucocorticoid-induced TNF receptor). Unlike conventional T cells, Tregs do not produce IL-2 and are therefore anergic at baseline.

The term “natural Treg” means a thymus-derived Treg. Natural Tregs are CD4+CD25+FOXP3+Helios+Neuropilin1+. The term “natural Treg” distinguishes thymus-derived Tregs from “induced Tregs”, which develop from conventional T cells outside the thymus. Compared with induced Tregs, natural Tregs have higher expression of PD-1 (programmed cell death-1, pdcd1), neuropilin 1 (Nrp1), Helios (Ikzf2), and CD73. Natural Tregs may be distinguished from induced Tregs on the basis of the expression of Helios protein or Neuropilin 1 (Nrp1) individually.

As used herein, the term “induced regulatory T cell” (iTreg) means a CD4+CD25+FOXP3+Helios−Neuropilin 1−T cell which develops from mature CD4+ conventional T cells outside of the thymus. For example, iTregs can be induced in vitro from CD4+CD25−FOXP3− cells in the presence of IL-2 and TGF-β. Suitably, the Treg expresses FOXP3 from the endogenous FoxP3 gene of the cell. Suitably, the Treg may be a CD4+CD25+FOXP3+Treg. Suitably, the Treg may be a CD4+CD25+CD127−Treg.

Suitably, the Treg may be a CD4+CD25+CD127low Treg. Suitably, the Treg may be a CD4+CD25+CD127−CD45RA+Treg. Suitably, the Treg may be a CD4+CD25+CD127lowCD45RA+Treg. Suitably, the Treg may be a CD4+CD25+FOXP3+CD127−Treg. Suitably, the Treg may be a CD4+CD25+FOXP3+CD127low Treg. Suitably, the Treg is a CD4+CD25+FOXP3+Helios+Treg. Suitably, the Treg is a CD4+CD25+FOXP3+Neuropilin 1+Treg. Suitably, the Treg is a CD4+CD25+FOXP3+Helios+Neuropilin 1+Treg. Suitably, the Treg is a human Treg. Suitably the Treg is a human Treg and the FOXP3 is human FOXP3. The term “Tconv cells”, meaning conventional T cells, refers to T cells that are not Tregs. In one aspect, the Treg of the present invention may be derived from a stem cell. In particular, the Treg of the present invention may be derived from a stem cell in vitro. In another aspect, the cell is a progenitor cell.

As used herein, the term “stem cell” means an undifferentiated cell which is capable of indefinitely giving rise to more stem cells of the same type, and from which other, specialised cells may arise by differentiation. Stem cells are multipotent. Stem cells may be for example, embryonic stem cells or adult stem cells.

As used herein, the term “progenitor cell” means a cell which is able to differentiate to form one or more types of cells but has limited self-renewal in vitro. Suitably, the cell is capable of being differentiated into a T cell, such as a Treg. Suitably, the cell has the ability to differentiate into a T cell, which expresses FOXP3 such as a Treg. Suitably, the cell may be an embryonic stem cell (ESC). Suitably, the cell is a haematopoietic stem cell or haematopoietic progenitor cell. Suitably, the cell is an induced pluripotent stem cell (iPSC). Suitably, the cell may be obtained from umbilical cord blood. Suitably, the cell may be obtained from adult peripheral blood.

In some aspects, hematopoietic stem and progenitor cell (HSPCs) may be obtained from umbilical cord blood. Cord blood can be harvested according to techniques known in the art (e.g., U.S. Pat. Nos. 7,147,626 and 7,131,958 which are incorporated herein by reference). In one aspect, HSPCs may be obtained from pluripotent stem cell sources, e.g., induced pluripotent stem cells (iPSCs) and embryonic stem cells (ESCs).

As used herein, the term “hematopoietic stem and progenitor cell” or “HSPC” refers to a cell which expresses the antigenic marker CD34 (CD34+) and populations of such cells. In particular embodiments, the term “HSPC” refers to a cell identified by the presence of the antigenic marker CD34 (CD34+) and the absence of lineage (lin) markers. The population of cells comprising CD34+ and/or Lin(−) cells includes haematopoietic stem cells and hematopoietic progenitor cells. HSPCs can be obtained or isolated from bone marrow of adults, which includes femurs, hip, ribs, sternum, and other bones. Bone marrow aspirates containing HSPCs can be obtained or isolated directly from the hip using a needle and syringe. Other sources of HSPCs include umbilical cord blood, placental blood, mobilized peripheral blood, Wharton's jelly, placenta, fetal blood, fetal liver, or fetal spleen. In particular embodiments, harvesting a sufficient quantity of HSPCs for use in therapeutic applications may require mobilizing the stem and progenitor cells in the subject.

The term “immune response” refers to a number of physiological and cellular effects facilitated by the immune system in response to a stimulus such as a pathogen or an autoantigen. Examples of such effects include increased proliferation of Tconv cells and secretion of cytokines. Any such effects may be used as indicators of the strength of an immune response. A relatively weaker immune response by Tconv in the presence of modified Tregs compared to non-modified Treg would indicate a relative enhancement of the modified Tregs to suppress immune responses. For example, a relative decrease in cytokine secretion would be indicative of a weaker immune response, and thus an enhancement of the ability of Tregs to suppress immune responses. Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs. Antigen-specific Tconv cells co-cultured with Tregs of the present invention having increased FOXP3 expression may proliferate 5, 10, 15, 20, 25, 30, 35 or 40% less than the same Tconv cells co-cultured with corresponding Tregs that do not have increased FOXP3 expression. Antigen-specific Tconv cells co-cultured with Tregs of the invention having increased FOXP3 expression may show a reduction of effector cytokine that is at least 10%, at least 20%, at least 30%, at least 40%, at least 50%, or at least 60% greater than corresponding Tconv cells co-cultured with corresponding Tregs that do not have increased FOXP3 expression. Antigen-specific Tconv cells co-cultured with Tregs of the invention having increased FOXP3 expression may produce 10%, 20%, 30%, 40%, 50%, 60% or less effector cytokine than than corresponding Tconv cells co-cultured with corresponding Tregs that do not have increased FOXP3 expression. The effector cytokine may be selected from IL-2, IL-17, TNFα, GM-CSF, IFN-γ, IL-4, IL-5, IL-9, IL-10 and IL-13. Suitably the effector cytokine may be selected from IL-2, IL-17, TNFα, GM-CSF and IFN-γ. Antigen-specific Tconv cells co-cultured with Tregs of the invention having increased FOXP3 expression may achieve suppression of IL-2 production at ½, ¼, ⅛, 1/10 or 1/20 the cell number of corresponding Tregs that do not have increased FOXP3 expression.

As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell (HSC and HPC respectively).

As used herein, the term “reprogramming” refers to a method of increasing the potency of a cell to a less differentiated state.

As used herein, the term “programming” refers to a method of decreasing the potency of a cell or differentiating the cell to a more differentiated state.

In one embodiment of the invention there is provided a method of enhancing T regulatory cell-cell homing and/or retention potential, the method comprising of; a) obtaining a selected cell population enriched for T regulatory cells; b) expanding the selected cell population with conditions suited for selective proliferation of FoxP3 expressing cells; b providing nicotinamide in the range of 0.1 to 500 mM for a period of time sufficient for enhancing T regulatory cells homing and/or retention potential, thereby enhancing homing and/or retention potential of T regulatory cells in the selected cell population. In one embodiment of the invention, disclosed is a method of increasing T regulatory cell mobility by augmenting expression of CD62 ligand. This upregulation may be accomplished by culturing of the cells or progenitor cells in a hypoxic environment. In some embodiments the hypoxia is administered in a manner to increase expression of hypoxia inducible factor (HIF-1). In other embodiments the invention provides the upregulation of homing molecules such as HIF-1 through enriching T regulatory cells and providing nicotinamide in the range of 0.1 to 500 mM for a period of time sufficient for enhancing T regulatory cell CD62L expression, thereby enhancing CD62L expression of T regulatory cell in the selected cell population. According to still further features in the described preferred embodiments the conditions for T regulatory cell expansion comprise providing nutrients and cytokines. According to still further features in the described preferred embodiments the cytokines are selected from the group consisting of IL-2, IL-15 and IL-21. According to still further features in the described preferred embodiments the nicotinamide is selected from the group consisting of nicotinamide, a nicotinamide analog, a nicotinamide metabolite, a nicotinamide analog metabolite and derivatives thereof. According to still further features in the described preferred embodiments the selected cell population is a lymphocyte cell population enriched for T regulatory by selection of FoxP3 positive cells. According to still further features in the described preferred embodiments providing the conditions for T regulatory cell expansion and the nicotinamide enhances homing and/or retention potential and/or CD62L expression of the T regulatory cells producing IL-10 in the selected cell population.

In another aspect, provided herein is an ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells, wherein the ex vivo-expanded Treg cell population has been expanded from baseline Tregs, and wherein, in the ex vivo-expanded Treg cell population, expression of one or more Treg-associated signature gene products listed in is increased relative to the expression of the one or more gene products in baseline Tregs. In some embodiments, in the ex vivo-expanded Treg cell population expression of one or more dysfunctional baseline signature gene is decreased relative to the expression of the one or more gene products in baseline Tregs. In some embodiments, the ex vivo-expanded Treg cell population exhibits a suppressive function, wherein the suppressive function is at least 50%, at least 60%, at least 70%, at least 75%, at least 80%, at least 85%, at least 90% or at least 95% as determined by suppression of proliferation of responder T cells by flow cytometry or thymidine incorporation. In some embodiments, the ex vivo-expanded Treg cell population exhibits a suppressive function, wherein the suppressive function is at least 50%, at least 75%, at least 100%, or at least 150% that of baseline Tregs, as determined by suppression of proliferation of responder T cells by flow cytometry or thymidine incorporation.

The invention provides T regulatory cells that may be generated from pluripotent sources as natural T regulatory cells or induced T regulatory cells which developed from progenitor cells that originated from pluripotent stem cells, or in some cases from expanded conventional T cells. The T regulatory cells of the invention are enhanced for increased mobility and migration activity through alteration of the cytoskeleton. In some means of practicing the invention suppression of cytoskeleton proteins is achieved in T regulatory cells. This suppression, whether permanent through technologies such as gene editing, or temporary, through short interfering RNA or antisense oligonucleotides achieves enhanced deformability of the T regulatory cell, which allows for enhanced migration of the T regulatory cell to inflammatory stimuli. Numerous inflammatory stimuli are known to cause migration of T regulatory cells, however in many cases the stiffness of the cell membrane does not allow for homing of the cells to properly inhibit said inflammation.

In the practices of the invention, in one embodiment the T regulatory cells of the present invention are natural T regulatory cells or induced T regulatory cells which developed from conventional T cells in vivo. Suitable Treg cells include thymus-derived, natural Treg (nTreg) cells and peripherally generated, induced Treg (iTreg) cells. In other words, the T regulatory cells of the present invention express endogenous FOXP3. Surprisingly, the present inventors have determined that increasing AIRE expression in Tregs which already express endogenous FOXP3 (e.g. by introducing exogenous AIRE) enhances the regulatory function of the T regulatory cells to a greater degree than the regulatory function provided by expressing exogenous FOXP3 in conventional T cells which do not express endogenous FOXP3. Additionally, transfection of T cells whether derived naturally, or derived from pluripotent stem cells, with agents that suppress cytoskeletal proteins such as shRNA to vimentin, leads to generation of T regulatory cells which possess enhanced migration.

Other means of increasing migration potency of T regulatory cells includes transfection of said T regulatory cells will chemokine receptors. Various chemokine receptors are useful for this purpose. In some cases transfection CXCR4, which is the receptor for SDF-1, otherwise known as CXCL12 is utilized to augment migration. SDF-1 is produced by areas of hypoxia in the tissue, and this is usually associated with tissue injury. In one embodiment the present inventors have further determined that increasing AIRE expression together with CXCR4 in T regulatory cells which already express endogenous FOXP3 enables improved retention of a T regulatory cells functional profile in vivo following administration to a subject. For example, it has been determined that natural T regulatory cells which do not express exogenous FOXP3 may lose their T regulatory cells profile following administration to a subject-for example natural T regulatory cells which do not express exogenous FOXP may have reduced levels of FOXP3 expression and be capable of producing pro-inflammatory, effector cytokines after a period following administration to a subject. T regulatory cells provided by the present invention may retain FOXP3 expression and have reduced capability to produce pro-inflammatory, effector cytokines after a period following administration to a subject. The utilization of enhanced migrating T regulatory cells for treatment of numerous conditions is envisioned through numerous mechanisms. In one example T regulatory cells overexpressing CXCR3 are administered in a patients with rheumatoid arthritis. Said T regulatory cells are useful due to enhanced ability to home into the synovium. In some embodiment the T regulatory cells are transfected with antioxidant genes to enhance ability to survive in the harsh microenvironment associated with inflammation. In some embodiments enhancement of T regulatory cell survival is accomplished by transfection with antioxidant genes such as superoxide dismutase, catalase or xanthine oxidase. The transfection of said T regulatory cells can occur at the level of the T regulatory cell progenitor or at the level of pluripotent stem cell. In one aspect, the invention provides a method for generating a population of regulatory T cells (Tregs) comprising providing a first population of Tregs and increasing vimentin suppression in the first population of Tregs to generate a second population of Tregs. The invention provides a method for enhancing the ability of a Tregs to suppress immune responses comprising decreasing vimentin expression in the Treg. In some embodiments of the invention, FOXP3 expression is increased by introducing into the Tregs a polynucleotide encoding a FOXP3 protein. In some embodiments of the invention, the method for enhancing the ability of regulatory T cells (Tregs) to suppress immune responses comprises: (a) isolating a Treg from a cell population; and (b) decreasing vimentin expression in Tregs. Suitably, the Treg may refer to a population of Tregs (i.e. a plurality of Tregs). The invention also provides an engineered Treg obtainable or obtained by the method of the invention. The invention also provides an engineered Treg having higher FOXP3 expression than a non-engineered Treg. In some embodiments the engineered Treg comprises an exogenous polynucleotide blocking expression of vimentin protein. The invention also provides a pharmaceutical composition comprising an engineered Treg of the invention. The invention also provides an engineered Treg of the invention or a pharmaceutical composition of the invention for use in prevention and/or treatment of a disease. The invention also provides the use of an engineered Treg of the invention in the manufacture of a medicament. The invention also provides a method for prevention and/or treatment of a disease comprising administering to a subject an engineered Treg or a composition of the invention.

According to still further features in the described preferred embodiments the selected cell population is a lymphocyte cell population enriched for T regulatory cells expressing FoxP3. According to still further features in the described preferred embodiments the selected cell population is a lymphocyte cell population enriched for T regulatory cells expressing IL-10 receptor. According to still further features in the described preferred embodiments the selected cell population is a lymphocyte cell population enriched for T regulatory cells expressing CTLA4. According to still further features in the described preferred embodiments the selected cell population is a lymphocyte cell population enriched for T regulatory cells expressing TGF-beta. According to still further features in the described preferred embodiments the selected cell population is a lymphocyte cell population enriched for T regulatory cells expressing latency associated protein. According to still further features in the described preferred embodiments the selected cell population is a lymphocyte cell population enriched for T regulatory cells expressing GITR. According to still further features in the described preferred embodiments the selected cell population is a lymphocyte cell population enriched for T regulatory cells expressing BTLA4.

According to still further features in the described preferred embodiments the population of T regulatory cells is derived from an organ selected from the group consisting of a muscle, skin, a bone, a lymph organ, a pancreas, a liver, a gallbladder, a kidney, a digestive tract organ, a respiratory tract organ, a reproductive organ, a urinary tract organ, a blood-associated organ, a thymus, a spleen, a nervous system organ According to still further features in the described preferred embodiments the population of T regulatory cell is derived from a source selected from the group consisting of hematopoietic cells, umbilical cord blood cells, mobilized peripheral blood cells and bone marrow cells. According to still further features in the described preferred embodiments the population of T regulatory cell is derived from bone marrow or peripheral blood. According to still further features in the described preferred embodiments the population of T regulatory cell is derived from neonatal umbilical cord blood. According to still further features in the described preferred embodiments the population of cells is derived from a mononuclear cell fraction. According to still further features in the described preferred embodiments the population of T regulatory cell is from an apheresis sample. According to still further features in the described preferred embodiments the period of time of exposing T cells to nicotinamide of the method is between 1 and 3 weeks. According to still further features in the described preferred embodiments the period of time of exposing T cells to nicotinamide of the method is between 1 and 7 days. According to still further features in the described preferred embodiments the concentration of the nicotinamide is in the range of 0.5-20 mM. According to still further features in the described preferred embodiments the nicotinamide is provided at a concentration of 5 mM. According to still further features in the described preferred embodiments the method further comprising selecting a T regulatory cell population according to a cell marker selected from the group consisting of a tumor antigen, a viral antigen and a bacterial antigen.

In some embodiments the invention discloses the use of enhanced Treg cells for treatment of autoimmune disease. Said enhanced Treg cells possess augmented ability to migrate to areas of inflammation and autoimmunity. Autoimmune diseases that may be treated by the current invention include chlorhydra autoimmune active chronic hepatitis, acute disseminated encephalomyelitis, acute hemorrhagic leukoencephalitis, Addison's disease, agammaglobulinemia, alopecia areata, Alzheimer's disease, amyotrophic lateral sclerosis, ankylosing spondylitis, anti-gbm/tbm nephritis, antiphospholipid syndrome, antisynthetase syndrome, aplastic anemia, arthritis, atopic allergy, atopic dermatitis, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune polyendocrine syndrome, autoimmune progesterone dermatitis, autoimmune thrombocytopenia purpura, autoimmune uveitis, balo disease/balo concentric sclerosis, Bechets syndrome, Berger's disease, Bickerstaff's encephalitis, Blau syndrome, bullous pemphigoid, Castleman's disease, Chagas disease, chronic fatigue immune dysfunction syndrome, chronic inflammatory demyelinating polyneuropathy, chronic lyme disease, chronic obstructive pulmonary disease, Churg-Strauss syndrome, cicatricial pemphigoid, coeliac disease, Cogan syndrome, cold agglutinin disease, cranial arteritis, crest syndrome, Crohns disease, Cushing's syndrome, Dego's disease, Dercum's disease, dermatitis herpetiformis, dermatomyositis, diabetes mellitus type 1, Dressler's syndrome, discoid lupus erythematosus, eczema, endometriosis, enthesitis-related arthritis, eosinophilic fasciitis, epidermolysis bullosa acquisita, essential mixed cryoglobulinemia, Evan's syndrome, fibrodysplasia ossificans progressive, fibromyalgia, fibromyositis, fibrosing aveolitis, gastritis, gastrointestinal pemphigoid, giant cell arteritis, glomerulonephritis, Goodpasture's syndrome, Graves' disease, Guillain-barré syndrome (GBS), Hashimoto's encephalitis, Hashimoto's thyroiditis, henoch-schonlein purpura, hidradenitis suppurativa, Hughes syndrome, inflammatory bowel disease (IBD), idiopathic inflammatory demyelinating diseases, idiopathic pulmonary fibrosis, idiopathic thrombocytopenia purpura, iga nephropathy, inflammatory demyelinating polyneuopathy, interstitial cystitis, irritable bowel syndrome (IBS), Kawasaki's disease, lichen planus, Lou Gehrig's disease, lupoid hepatitis, lupus erythematosus, ménière's disease, microscopic polyangiitis, mixed connective tissue disease, morphea, multiple myeloma, multiple sclerosis, myasthenia gravis, myositis, narcolepsy, neuromyelitis optica, neuromyotonia, occular cicatricial pemphigoid, opsoclonus myoclonus syndrome, ord thyroiditis, Parkinson's disease, pars planitis, pemphigus, pemphigus vulgaris, pernicious anaemia, polymyalgia rheumatic, polymyositis, primary biliary cirrhosis, primary sclerosing cholangitis, progressive inflammatory neuropathy, psoriasis, psoriatic arthritis, raynaud phenomenon, relapsing polychondritis, Reiter's syndrome, rheumatoid arthritis, rheumatoid fever, sarcoidosis, schizophrenia, Schmidt syndrome, Schnitzler syndrome, scleritis, scleroderma, Sjögren's syndrome, spondyloarthropathy, sticky blood syndrome, still's disease, stiff person syndrome, sydenham chorea, sweet syndrome, takayasu's arteritis, temporal arteritis, transverse myelitis, ulcerative colitis, undifferentiated connective tissue disease, undifferentiated spondyloarthropathy, vasculitis, vitiligo, Wegener's granulomatosis, Wilson's syndrome, Wiskott-Aldrich syndrome, hypersensitivity reactions of the skin, atherosclerosis, ischemia-reperfusion injury, myocardial infarction, and restenosis.

According to one aspect of the present invention there is provided a therapeutic cell composition comprising an expanded selected T regulatory cell population, the cell population ex-vivo cultured with conditions for T regulatory cell expansion and amount of nicotinamide in the range of 0.5-50 mM, wherein the expanded selected T regulatory cell population is characterized by at least one of: (i) enhanced T regulatory cell homing and/or retention potential, and (ii) enhanced expression of CD62L, as compared to a similar selected T regulatory cell population expanded with identical conditions and no more than 0.10 mM nicotinamide. According to still further features in the described preferred embodiments the therapeutic cell composition comprises T regulatory cell cultured according to the method of the invention as detailed herein.

According to still further features in the described preferred embodiments the subject is being treated with umbilical cord blood hematopoietic stem cells expanded in culture with greater than 1.0 mM nicotinamide prior to, concomitantly with or following transplantation of the expanded Treg cells.

In one embodiment of the invention T regulatory cells are generated from iPSC cells and said T regulatory cells are treated in a manner to induce dedifferentiation. This increases homing ability of T regulatory cells towards chemotactic gradients. In some embodiments T regulatory cells are treated with inhibitors of vimentin cytoskeletal assembly. This provides an increased flexibility of said T regulatory cell cytoskeleton, thus allowing for increased access of said T regulatory cells to different tissues. In one embodiment the invention provides generation of T regulatory cells possessing a more deformable cytoskeleton as compared to conventional T regulatory cells. In another embodiment the invention provides ability of the modified T regulatory cell to penetrate tissues in part through transfection of said T regulatory cells with matrix metalloproteases, thus allowing for entry and migrations through tissues.

In some embodiments, the method according to the invention comprises:
(a) isolating the Treg from a differentiating cell population; and
(b) Decreasing expression of one or more cytoskeletal proteins in the Treg.

The expression “isolating the Treg from a cell population” means to separate out the Treg from a heterogeneous mixture of multiple different types of cells. Suitable the cell population is from a sample from a human subject. Suitably, the Treg is isolated as a population of Tregs. Suitably, the population of Tregs comprises at least 70% Tregs, such as 75%, 85%, 90% or 95% Tregs. In some embodiments of the invention, the cell population comprises or consists of peripheral blood mononuclear cells (PBMCs). A PBMC is any blood cell with a round nucleus found within the circulating pool of blood, rather than sequestered in the bone marrow, liver, spleen or lymphatic system. PBMCs consist of monocytes and lymphocytes (T cells, B cells and NK cells). Techniques for isolation of PBMCs from whole blood are known in the art. For example, PBMCs can be separated from a blood sample by addition of a density gradient medium, such as Ficoll (GE Healthcare), followed by centrifugation. The different types of cells in the blood separate out into different layers, including a layer containing the PBMCs. In some embodiments of the invention, isolating the Treg comprises isolating CD4+ T cells. In some embodiments, isolating the Treg comprises isolating CD4+ T cells and subsequently isolating the Treg from the CD4+ T cells. CD4 (cluster of differentiation 4) is a co-receptor of the T cell receptor expressed by various types of T cells. Isolation of CD4+ cells separates T cells, including Tregs, from the initial cell population. The Tregs may then be isolated from this T cell-enriched population. Techniques for isolating specific cell types from a heterogeneous population of cells are known in the art. Examples include use of immuno-magnetic beads and fluorescence-activated cell sorting. In some embodiments of the invention, isolating the population of Tregs comprises using immuno-magnetic beads. Various companies (e.g. Miltenyi Biotec, Stem Cell Technologies, ThermoFisher Scientific) offer kits comprising immuno-magnetic beads for isolation of specific types of T cells (see, for example, Fallarino et al. (2003) Modulation of tryptophan catabolismby regulatory T cells. Nat. Immunol. 4:1206-1212, incorporated herein by reference). These isolation kits make use of antibodies widely available in the art to T cell surface proteins such as CD8, CD25, CD49b and others. For example, CD4+ cells may be first negatively selected by incubating the cell population with biotin-conjugated antibodies to markers of non-CD4+ cells (e.g. CD8) and removing these cells using anti-biotin magnetic beads. Then, Tregs may be positively selected by incubation with anti-CD25-labelled beads. In some embodiments of the invention, isolating the population of Tregs comprises fluorescence-activated cell sorting (FACS). In some embodiments, the Tregs are sorted according to their CD4+CD25hiCD127− phenotype.

Natural Tregs may be sorted from induced Tregs on the basis of expression of Helios protein or Neuropilin 1. In some embodiments of the invention, the natural Tregs may be sorted according to their CD4+CD25+FOXP3+Helios+Neuropilin1+phenotype. FACS is a form of flow cytometry which is well-known in the art. During FACS, cells are suspended in fluid and streamed through a detection system that analyses various characteristics. Cells can be sorted according to their characteristics using this method. In particular, in FACS, molecules are marked using fluorescent antibodies and cells sorted according to their degree of fluorescence, which indicates level of expression of the particular molecule (see Adan et al. Flow cytometry: basic principles and applications Crit. Rev. Biotechnol. 2017 March; 37 (2): 163-176, incorporated herein by reference).

In certain embodiments, the present disclosure encompasses administration of T regulatory cells treated with a vimentin inhibitor for treatment and/or prevention of type 1 diabetes. In some embodiments said T regulatory cells are administered together with anti-human CD3 antibodies such teplizumab to individuals predisposed to develop type 1 diabetes or with pre-clinical stages of type 1 diabetes, but who do not meet the diagnosis criteria as established by the American Diabetes Association or the Immunology of Diabetes Society to prevent or delay the onset of type 1 diabetes and/or to prevent or delay the need for administration of insulin to such patients.

In certain embodiments, enhanced T regulatory cells alone or in combination with other agents are administered, agents useful for enhancing immune modulatory activity include nitric oxide synthase inhibitors and nitric oxide scavangers comprising; arginine derivatives, methylated arginines, substituted L-arginine, nitro-arginine, L-N.sup.G-nitroarginine, N.sup.G-mono-methyl-L-arginine (L-NMMA), N-nitro-L-arginine methyl ester (L-NAME), N-amino-L-arginine, N-methyl-L-arginine, N.sup.G-monomethyl-L-arginine (L-NMA), N.sup.G-nitro-L-arginine (L-NNA), aminoguanidine, 7-nitroindazole, S-ethylisothiourea, S-methylisothiourea, S-methylthiocitriulline, S-ethylthiocitrulline, N-ethylimino-L-ornithine, N-iminoethyl-L-lysine (L-NIL), flavoprotein binders. diphenyleneiodonium and related iodonium derivatives, omithine and omithine derivatives; tetracycline and derivaties thereof; L-canavanine; citrulline; redox dyes, methylene blue; calmodulin binders, trifluoropiperazine and calcinarin; heme binders; resveratrol; zinc compounds; tetrahydropterin analogs, aminoguanidine; and depleters of biopterin, methotrexate, N-acetylcysteine, nonsteroidal anti-inflammatory agents, sodium salicylate, and mixtures thereof.

Patients for which the invention may be applicable to include those suffering from high-risk factors for identification of predisposed subjects include having first or second degree relatives with diagnosed type-1 diabetes, an impaired fasting glucose level (e.g., at least one determination of a glucose level of 100-125 mg/dl after fasting (8 hour with no food)), an impaired glucose tolerance in response to a 75 g OGTT (e.g., at least one determination of a 2-hr glucose level of 140-199 mg/dl in response to a 75 g OGTT), an HLA type of DR3, DR4 or DR7 in a Caucasian, an HLA type of DR3 or DR4 in a person of African descent, an HLA type of DR3, DR4 or DR9 in a person of Japanese descent, exposure to viruses (e.g., coxsackie B virus, enteroviruses, adenoviruses, rubella, cytomegalovirus, Epstein-Barr virus), a positive diagnosis according to art accepted criteria of at least one other autoimmune disorder (e.g., thyroid disease, celiac disease), and/or the detection of autoantibodies, particularly ICAs and type 1 diabetes-associated autoantibodies, in the serum or other tissues. In certain embodiments, the subject identified as predisposed to developing type 1 diabetes has at least one of the risk factors described herein and/or as known in the art. The present disclosure also encompasses identification of subjects predisposed to development of type 1 diabetes, wherein said subject presents a combination of two or more, three or more, four or more, or more than five of the risk factors disclosed herein or known in the art.

In some embodiments patients are selected based on antibodies. Serum autoantibodies associated with type 1 diabetes or with a predisposition for the development of type 1 diabetes are islet-cell autoantibodies (e.g., anti-ICA512 autoantibodies), glutamic acid decarbamylase autoantibodies (e.g., anti-GAD65 autoantibodies), IA2 antibodies, ZnT8 antibodies and/or anti-insulin autoantibodies. Accordingly, in a specific example in accordance with this embodiment, the invention encompasses the treatment of an individual with detectable autoantibodies associated with a predisposition to the development of type 1 diabetes or associated with early stage type 1 diabetes (e.g., anti-IA2, anti-ICA512, anti-GAD or anti-insulin autoantibodies), wherein said individual has not been diagnosed with type 1 diabetes and/or is a first or second degree relative of a type-1 diabetic. In certain embodiments, the presence of the autoantibodies is detected by ELISA, electrochemoluminescence (ECL), radioassay (see, e.g., Yu et al., 1996, J. Clin. Endocrinol. Metab. 81:4264-4267), agglutination PCR (Tsai et al, ACS Central Science 2016 2 (3), 139-147) or by any other method for immunospecific detection of antibodies described herein or as known to one of ordinary skill in the art. B-cell function prior to, during, and after therapy may be assessed by methods described herein or by any method known to one of ordinary skill in the art. For example, the Diabetes Control and Complications Trial (DCCT) research group has established the monitoring of percentage glycosylated hemoglobin (HA1 and HA1c) as the standard for evaluation of blood glucose control (DCCT, 1993, N. Engl. J. Med. 329:977-986). Alternatively, characterization of daily insulin needs, C-peptide levels/response, hypoglycemic episodes, and/or FPIR may be used as markers of β-cell function or to establish a therapeutic index (See Keymeulen et al., 2005, N. Engl. J. Med. 352:2598-2608; Herold et al., 2005, Diabetes 54:1763-1769; U.S. Pat. Appl. Pub. No. 2004/0038867 A1; and Greenbaum et al., 2001, Diabetes 50:470-476, respectively). For example, FPIR is calculated as the sum of insulin values at 1 and 3 minutes post IGTT, which are performed according to Islet Cell Antibody Register User's Study protocols (see, e.g., Bingley et al., 1996, Diabetes 45:1720-1728 and McCulloch et al., 1993, Diabetes Care 16:911-915). In some embodiments, the individuals predisposed to develop T1D can be a non-diabetic subject who is a relative of a patient with T1D. In certain embodiments, the non-diabetic subject has 2 or more diabetes-related autoantibodies selected from islet cell antibodies (ICA), insulin autoantibodies (IAA), and antibodies to glutamic acid decarboxylase (GAD), tyrosine phosphatase (IA-2/ICA512) or ZnT8. In various embodiments, the non-diabetic subject has abnormal glucose tolerance on oral glucose tolerance test (OGTT). Abnormal glucose tolerance on OGTT is defined as a fasting glucose level of 110-125 mg/dL, or 2 hour plasma of ≥140 and <200 mg/dl, or an intervening glucose value at 30, 60, or 90 minutes on OGTT >200 mg/dL.

In some embodiments, the non-diabetic subject who will respond to the Treg cells does not have antibodies against ZnT8. In certain embodiments, such non-diabetic subject is HLA-DR4+ and is not HLA-DR3+. In some embodiments, such non-diabetic subject who will respond to the anti-CD3 antibody such as teplizumab demonstrates an increase, following administration (e.g., after 1 month, after 2 months, after 3 months, or longer or shorter), in the frequency (or relative amount) of TIGIT+KLRG1+CD8+ T-cells (e.g., by flow cytometry) in peripheral blood mononuclear cells. In various embodiments, the prophylactically effective amount comprises a 10 to 14 day course of subcutaneous (SC) injection or intravenous (IV) infusion of the Treg cells.

In certain embodiments, Treg are administered the course of dosing with the anti-CD3 antibody such as teplizumab can be repeated at 2 month, 4 month, 6 month, 8 month, 9 month, 10 month, 12 month, 15 month, 18 month, 24 month, 30 month, or 36 month intervals. In specific embodiments efficacy of the treatment with the anti-CD3 antibody such as teplizumab is determined as described herein or as is known in the art at 2 months, 4 months, 6 months, 9 months, 12 months, 15 months, 18 months, 24 months, 30 months, or 36 months subsequent to the previous treatment. In another embodiment, a subject is administered one or more unit doses of approximately 0.5-50 ug/kg, approximately 0.5-40 ug/kg, approximately 0.5-30 ug/kg, approximately 0.5-20 ug/kg, approximately 0.5-15 ug/kg, approximately 0.5-10 ug/kg, approximately 0.5-5 ug/kg, approximately 1-5 ug/kg, approximately 1-10 ug/kg, approximately 20-40 ug/kg, approximately 20-30 ug/kg, approximately 22-28 ug/kg or approximately 25-26 ug/kg of the anti-CD3 antibody such as teplizumab to prevent, treat or ameliorate one or more symptoms of T1D. In another embodiment, a subject is administered one or more unit doses of about 200 ug/kg, 178 ug/kg, 180 ug/kg, 128 ug/kg, 100 ug/kg, 95 ug/kg, 90 ug/kg, 85 ug/kg, 80 ug/kg, 75 ug/kg, 70 ug/kg, 65 ug/kg, 60 ug/kg, 55 ug/kg, 50 ug/kg, 45 ug/kg, 40 ug/kg, 35 ug/kg, 30 ug/kg, 26 ug/kg, 25 ug/kg, 20 ug/kg, 15 ug/kg, 13 ug/kg, 10 ug/kg, 6.5 ug/kg, 5 ug/kg, 3.2 ug/kg, 3 ug/kg, 2.5 ug/kg, 2 ug/kg, 1.6 ug/kg, 1.5 ug/kg, 1 ug/kg, 0.5 ug/kg, 0.25 ug/kg, 0.1 ug/kg, or 0.05 ug/kg of the anti-CD3 antibody such as teplizumab to prevent, treat or ameliorate one or more symptoms of T1D. In particular embodiments, a subject is administered one or more doses of the anti-CD3 antibody such as teplizumab at about 5-1200 ug/m2, preferably, 51-826 ug/m2. In another embodiment, a subject is administered one or more unit doses of 1200 ug/m2, 1150 ug/m2, 1100 ug/m2, 1050 ug/m2, 1000 ug/m2, 950 ug/m2, 900 ug/m2, 850 ug/m2, 800 ug/m2, 750 ug/m2, 700 ug/m2, 650 ug/m2, 600 ug/m2, 550 ug/m2, 500 ug/m2, 450 ug/m2, 400 ug/m2, 350 ug/m2, 300 ug/m2, 250 ug/m2, 200 ug/m2, 150 ug/m2, 100 ug/m2, 50 ug/m2, 40 ug/m2, 30 ug/m2, 20 ug/m2, 15 ug/m2, 10 ug/m2, or 5 ug/m2 of the anti-CD3 antibody such as teplizumab to prevent, treat, slow the progression of, delay the onset of or ameliorate one or more symptoms of T1D.

Treatment may be provided to the subject after clinical onset of the condition (e.g. type 1 diabetes). Clinical onset of type 1 diabetes may be the need for a subject to utilize insulin injections to regulate blood sugar levels. In pediatric patients, a blood sugar level below 70 mg/dl can be considered low and, in some instances, can be characterized by symptoms such as, e.g. sweating, hunger and/or shakiness. In pediatric patients, a blood sugar level above 200 mg/dl can be considered high and can be characterized, in some instances, by low energy, stomachaches, and/or difficulty breathing. In pediatric patients, about 70 to about 120 mg/dl blood sugar level is considered normal. In pediatric patients, about 120 to about 200 mg/dl blood sugar level is considered outside the normal range, but it can be within the goal or target range for pediatric patients trying to maintain blood sugar levels. For pediatric patients aged about 12 years and older, maintaining a blood sugar level from about 70 to about 150 mg/dl can be a goal (e.g. for pediatric patients with diabetes). For pediatric patients aged about five years of age to about eleven years of age, maintaining a blood sugar level from about 70 to about 180 mg/dl can be a goal (e.g. for pediatric patients with diabetes). For pediatric patients aged about five years of age or younger, maintaining a blood sugar level from about 80 to about 200 mg/dl can be a goal (e.g. for pediatric patients with diabetes). One skilled in the art will recognize that these ranges are standard guidelines and, e.g., individual target ranges may vary based on a patient's age, body size, development, and the like.

In the practice of the invention it is to be recognized that one treatment point is loss of insulin production. Clinical onset of type 1 diabetes may be hyperglycemia. Clinical onset of type 1 diabetes may be the inability for a subject to regulate blood glucose levels. Clinical onset of type 1 diabetes may be inflammation of the pancreas. Clinical onset of type 1 diabetes may be pancreatic beta cell autoimmunity. Clinical onset of type 1 diabetes may be partial destruction of pancreatic beta cell mass. Destruction of pancreatic beta cell mass may be inflamed tissue, expansion of fibrotic legions, cellular apoptosis, cellular necrosis, cellular loss of function (e.g. inability to produce insulin, reduced insulin production). Clinical onset of type 1 diabetes may be about: 99%, 95%, 90%, 85%, 80%, 75%, 70%, 65%, 60%, 55%, 50%, 45%, 40%, 35%, 30%, 25%, 20%, 15%, 10%, or 5% destruction of pancreatic beta cell mass. Clinical onset of type 1 diabetes may be more than about: 99%, 95%, 90%, 85%, 80%, 75%, 70%, 65%, 60%, 55%, 50%, 45%, 40%, 35%, 30%, 25%, 20%, 15%, 10%, 5% destruction of pancreatic beta cell mass. Clinical onset of type 1 diabetes may be less than about: 99%, 95%, 90%, 85%, 80%, 75%, 70%, 65%, 60%, 55%, 50%, 45%, 40%, 35%, 30%, 25%, 20%, 15%, 10%, 5% destruction of pancreatic beta cell mass. Clinical onset of type 1 diabetes may be complete destruction of pancreatic beta cell mass. Clinical onset of type 1 diabetes may include the onset of one or more symptoms of type 1 diabetes such as blurred vision, nausea, hyperglycemia, fatigue, weakness, muscle cramps, peripheral neuropathy, retinopathy, nephropathy, ulcers, other symptoms, and combinations thereof.

n certain aspects of the invention, the small compound glatiramer acetate (GA) (Copolymer 1/Copaxone) is used to modify dendritic cell function as to stimulate enhanced T regulatory cells. In another aspect, a small compound MAP kinase inhibitor is used to modify dendritic cell function as to enhance expression of FoxP3 in T regulatory cells. In yet another aspect, GA and a small compound MAP kinase inhibitor, such as, e.g., a c-Jun N-terminal kinase (JNK) small compound inhibitor, have a surprising synergistic effect on the modulation of dendritic cell function for the treatment or prevention of diabetes In some embodiments enhanced T regulatory cells are administered with tolerogenic dendritic cells. dendritic cells are pre-treated with inhibitors of NF-kappa b before their administration into tissue possessing degenerated beta cells. We describe the use of small molecules to regulate biological signals in order to alter the properties of dendritic cells. Signal regulation by small compounds (e.g., small molecule inhibitors) can control cell differentiation and function in a controllable and reproducible manner according to the current invention. The term “small compound” as used herein refers to compounds, chemicals, small molecules, small molecule inhibitors, or other factors that are useful for modulating dendritic cell function. Small molecule inhibitors have been used as immunosuppressive and anti-inflammatory drugs. GA (Copolymer 1/Copaxone) is an FDA approved drug for the treatment of T cell-mediated autoimmune disease. SP600125 is a small compound inhibitor of JNK, which is a downstream molecule of a number of signaling pathways that regulate both innate and adaptive immunity. The present invention is related to the discovery that these small compounds can regulate the suppressive functions of dendritic cell to facilitate the establishment of immune tolerance mediated by T regulatory cells with suppressed vimentin. In one embodiment of the invention GA is administered systemically as a treatment of diabetes and/or pancreatic degeneration together with enhanced T regulatory cells. In another embodiment treatment of islet rejection by administration of enhanced T regulatory cells alone or together with GAis disclosed. It has been known for a while that GA alone has not been effective for treating autoimmune diseases. Specifically, GA is known to be only partially effective for treating autoimmune conditions targeting endocrine organs Johnson et al. (1995) Neurology 45:1268-1276]. Moreover, clinical studies using GA for the treatment of IBD were discontinued, because GA failed to treat IBD. The present invention is based on the discovery that administration of GA in combination with dendritic cells, or with dendritic cells and a MAP kinase inhibitor (e.g., SP600125) under the cover of enhanced T regulatory cell suppression is surprisingly effective for the treatment T cell mediated autoimmunity of endocrine conditions such as type 1 diabetes,

In one embodiment of the invention vimentin suppressed T regulatory cells are utilized to expand populations of myeloid derived suppressor cells for induction of tolerance in the situation of autoimmunity or allogeneic immunity in the context of transplantation. Herein we use the term “myeloid-derived suppressor cell (MDSC)” to refer to an immature myeloid cell which is present in an immature state because of a granulocyte or the like not completely differentiated in tumors, autoimmune diseases, and infections, and it was reported that the number of MDSCs increases in patients with an acute inflammatory disease, trauma, septicemia, or a parasitic or mycotic infection, as well as in cancer patients. The generation of MDSC is thought to be part of the body's defense mechanisms against excessive inflammation and autoimmunity. This is known in part based on studies in which administration of MDSC can have therapeutic effects in conditions of autoimmunity and inflammation but conversely have negative effects in the context of neoplasia and/or viral and/or intracellular bacterial infections.

It is widely accepted in the literature that the function of MDSCs is to effectively suppress activated T cells. It is known that the mechanism by which MDSCs regulate T cells is that a nitric oxide synthase, reactive oxygen species (ROS), and arginase which are an enzyme suppress T cell activation by maximizing the metabolism of L-arginine which is an essential amino acid.

The current invention teaches the utilization of T regulatory cells in general, and specifically T regulatory cells from iPSC cells that have been generated to possesses suppressed levels of vimentin to augment the production of MDSCs. In some embodiments of the present invention, MDSCs are induced by T regulatory cells to differentiate from the CD34+ cells isolated from cord blood, may be monocytic myeloid-derived suppressor cells expressing cellular phenotypes of Lin−, HLA−DRlow, and CD11b+CD33+. In other embodiments said MDSCs can be generated in vivo by contact of T regulatory cells with naturally occurring MDSC precursors or stem cells. MSDCs that are generated by T regulatory cells may express PDL-1, CCR2, CCR5, CD62L, CXCR4, and ICAM-1 as cell surface markers.

According to an embodiment of the present invention, T regulatory cells and said cells subjected to means and method of enhancement may be utilized to generated MSDC for a variety of uses. Specifically, it may be the case that generation of MSDCs in vitro is desired, in which case CD34+ cells isolated from cord blood are cultured with T regulatory cells for 6 weeks and the cell surface thereof was stained, 70% HLA-ABC, 30% or less HLA-DR, and at least 90% CD45 are expressed, and compared to MDSCs whose differentiation was induced by exposure at a one to one ratio to T regulatory cells. 10% expression of CD83 and CD80 were observed only in the MDSCs whose differentiation was induced T regulatory cells according to the present invention. CD86 was expressed at about 40% in the MDSCs, which showed an aspect of low expression of co-stimulatory molecules. In addition, CD40 was expressed at 40%, and CD1d, CD3, and B220, which are lymphocyte markers, were expressed at less than 5%. PDL-1 which is known to suppress the proliferation or activation of T cells was expressed at about 30% only in cells cultured in the GM-CSF/SCF combination. CD13 is a transmembrane glycoprotein which is expressed in a myeloid precursor, myeloperoxidase (MPO) is a protein in azurophilic granules of myeloid cells, and both are proteins which are expressed in MDSCs. The expression of CD13 was significantly increased in MDSCs induced by a T regulatory cells compared to MDSCs induced by conventional T cells. MPO was expressed at 90% or more in all of the MDSCs induced by two different combinations.

In addition, MDSCs induced by T regulatory cells increase the expression of an immune suppressor substance selected from the group consisting of arginase 1, indoleamine 2,3-dioxygenase (IDO), and inducible nitric oxide synthase (iNOS), compared to MDSCs induced by a combination of conventional T cells and human peripheral blood-derived dendritic cells. MDSCs induced by the combination of T regulatory cells significantly suppress the proliferation of allogeneic CD4 T cells and thereby strongly reduce the secretion of IFN-γ by antigen-specific T cell immune responses. It was observed that MDSCs induced by the combination of T regulatory cells showed a significant increase in the secretion of IL-10 when stimulated with CD40 antibodies, and large amounts of VEFG and TGF-β were secreted without being affected by whether or not stimulated with the CD40 antibodies.

When CD4 T cells are stimulated by MDSCs in vitro, it is known that the number of Treg cells expressing FoxP3 increases, and when CD4 T cells are stimulated by MDSCs induced by T regulatory cells, FoxP3 expression is confirmed, but IL-17 which is an inflammatory cytokine is not secreted. In addition, MDSCs induced by the combination of T regulatory cells alleviate the degree of graft-versus-host disease, increases the survival rate, increases the secretions of serum anti-inflammatory cytokines, IL-10, and TGF-0, increases the secretions of anti-inflammatory proteins, CRP, MIP-3β, MMP-9, RANTES (CCL5), and SDF-1a, and suppresses inflammatory responses by reducing the secretions of inflammatory cytokines, IL-17, and IFN-γ, in an animal model for graft-versus-host disease. Moreover, the number of Treg cells expressing FoxP3 is increased. Therefore, the present invention provides a myeloid-derived suppressor cell, which is differentiated from a cord blood-derived CD34+ cell and proliferated, expresses cellular phenotypes of Lin−, HLA−DRlow, and CD11b+CD33+, and expresses PDL-1, CCR2, CCR5, CD62L, CXCR4, and ICAM-1 as cell surface markers.

In addition, the present invention provides an immunosuppressive composition, including the myeloid-derived suppressor cell which is monocytic stimulated by a T regulatory cell absent or low in vimentin expression. The myeloid-derived suppressor cell generated by enhanced T regulatory cells may be used to prevent or treat a rejection response in organ transplantation or hematopoietic stem cell transplantation; an autoimmune disease; or an allergic disease, which is caused by a hypersensitive immune response. In one embodiment the invention teaches the generation of “classical” MDSC from CD34 and/or CD133 myeloid lineage cells by contain with T regulatory cells and/or factors produced by said T regulatory cells. In another embodiment the invention teaches the generation of another distinct group of myeloid precursor cells that are defined as Lin−HLA−DR−CD33+ early-stage MDSCs which, however, only account for up to 5% of the total MDSCs population. In transplantation, alterations in MDSC subsets are rarely reported. The invention provides means of delaying and/or completely preventing transplant rejection and/or autoimmunity by creation of in vitro, ex vivo, or in vivo T regulatory cells.

MDSCs that are generated by the invention function to suppress and/or modulate the immune response through several means. The invention provides the use T regulatory cells to enhance various aspects of MDSCs. These cells have been shown to exhibit their major immunosuppressive effects through inducible nitric oxide synthase (iNOS), immunosuppressive cytokines and cell-surface molecules. iNOS converts L-arginine to nitric oxide (NO), which impairs T-cell proliferation by inducing apoptosis, suppressing T-cell mitogenic responses or inhibiting major histocompatibility complex class Il expression. In addition, MDSCs up-regulate the expression of immunosuppressive cytokines such as IL-10 and transforming growth factor-β, as well as immune regulatory cell-surface molecules like membrane-bound programmed death receptor ligand 1. There are other types of MDSCs which exert their suppressive effects on immune responses promoting the generation of reactive oxygen species (ROS) and arginase 1. In detail, MDSCs express NADPH oxidase 2 (Nox2) catalyzing the production of ROS, whereas increased expression of arginase-1 causes a reduction of local L-arginine and cysteine levels, which constitute a key nutrition for T-cell proliferation and function. Noteworthy, the described dichotomy does not imply an exclusive gene expression of either iNOS or arginase-1.

In some embodiments of the invention T regulatory cells are generated in a manner to enhance the timing of contact between said T regulatory cell and target cell which differentiate into MDSCs. In some embodiments said enhanced T regulatory cells increase the tolerogenic activities of said MDSCs through augmenting expression of iNOS and/or pathways downstream of interferon signaling. Synergic expression of iNOS and arginase-1 has namely been characterized to constitute a cardinal feature of the immunosuppressive function of MDSCs, supporting the generation of reactive nitrogen species. Reactive nitrogen species in turn impede protein-protein interactions and functions including chemotaxis, antigen recognition and activation of T cells. In addition, MDSCs appear to mediate the development of Treg that suppress T-cell responses against transplanted grafts via interferon (IFN)-γ-dependent pathways. IFN-γ seems to play a crucial role for tolerance induction in transplantation, as tolerance is not induced in IFN-γ-deficient recipient mice due to active CD28 and CD40 ligand T-cell co-stimulation pathways, resulting in the proliferation of alloreactive effector T lymphocytes. iNOS-expressing MDSCs seem essential for prolonging graft survival through stimulation of IFN-γ secretion in Tregs. In addition, CCL5 secreted by graft penetrating MDSCs has been shown to promote the accumulation of Tregs in tolerized kidney grafts. Conversely, the increase in Tregs augmented the number of MDSCs in the peripheral blood of the recipient, in the spleen and in the graft itself. Other mechanisms of MDSC-mediated immunosuppression that are mediated by both M-MDSCs and PMN-MDSCs include blockade of T-cell homing through reducing L-selectin expression on T cells, ectoenzyme-induced adenosine production and Fas/FasL-dependent apoptosis of cytotoxic T cells. Notably, a recent study showed a novel mechanism in which MDSC-derived methylglyoxal can be transferred to T cells via cell-cell transfer. Subsequently, methylglyoxal then paralyzed T-cell effector function through metabolic inhibition of these cells.

In one embodiment of the invention enhancement of immune modulatory activity is accomplished by transfection with genes known to be involved in immune modulation. For example it is known that MDSCs metabolize arginine though arginase-I and iNOS. Upon activation through IFN-γ, MDSCs have been shown to take up large amounts of Arg by inducing the cationic AA transporter 2, Arg-1 and iNOS. The reduction of local Arg levels in turn leads to decreased T-cell-derived CD37 expression, thus compromising T-cell antigen-specific proliferation. In addition, increased levels of NO released by iNOS in MDSCs further exert significant suppressive effects on T-cell function. Thus, MDSCs have been shown to inhibit alloreactive T-cell expansion via iNOS, thus promoting allograft survival of kidney transplants. Similarly, adoptive transfer of tumor necrosis factor-α-induced MDSCs prevented allograft rejection in male-to-female skin transplants through inhibiting T-cell proliferation that was abrogated in the presence of an NO-inhibitor or following genetic-depletion of NO. Notably, MDSCs and MDSCs display divergent features of Arg metabolism with MDSCs expressing greater levels of Arg-1, and MDSCs exhibiting higher levels of iNOS. In one embodiment T regulatory cells are transfected with Arg-1 and/or iNOS in order to augment immune modulatory activity of MDSCs. In another embodiment, T regulatory cells are transfected with Indoleamine 2, 3-dioxygenase (IDO). This is the key enzyme of tryptophan metabolism in the kynurenine (Kyn) pathway. MDSCs have been shown to exhibit an increased IDO expression and augmented Kyn pathway causing a metabolization of Trp from the surrounding microenvironment associated with decreased Trp levels. T cells in turn have been found highly sensitive for Trp shortage causing their cell-cycle arrest in the G1 phase, thus compromising T-cell-derived immune responses. Indeed, IDO expression was found to be critical, inhibiting T-cell-driven allo-immune responses with abrogated long-term cardiac allograft survival in CTLA4-Ig-treated mice deficient for IDO. Moreover, genetically induced IDO overexpression has been shown to suppress allograft rejection of cardiac allografts and small bowel transplants in mice through inhibiting T-cell-derived IFN-γ expression and increased Treg frequencies. In some embodiments CTLA-Ig is administered together with enhanced T regulatory cells for creation of tolerogenesis in conditions such as Type 1 Diabetes and Allograft Rejection. The immunosuppressive composition according to the present invention may further include a pharmaceutically acceptable carrier. Immune suppressive molecules useful for the invention are desired to possess some ability to maintain, if not actually promote, generation of T regulatory cells and initiation of a bidirectional look between said T regulatory cells and MDSC. Such molecules include FK-506 and rapamycin. The pharmaceutically acceptable carrier includes a carrier and a vehicle commonly used in the field of medicine, and specifically includes an ion exchange resin, alumina, aluminum stearate, lecithin, a serum protein (e.g., human serum albumin), buffer substances (e.g., various phosphates, glycine, sorbic acid, potassium sorbate, and partial glyceride mixtures of saturated vegetable fatty acids), water, salts or electrolytes (e.g., protamine sulfate, disodium hydrogen phosphate, potassium hydrogen phosphate, sodium chloride, and zinc salts), colloidal silica, magnesium trisilicate, polyvinyl pyrrolidone, cellulose-based substrates, polyethylene glycol, sodium carboxymethyl cellulose, polyarylates, wax, polyethylene glycol, wool grease, or the like, but the present invention is not limited thereto. In addition to the above components, the composition of the present invention may further include a lubricant, a wetting agent, an emulsifying agent, a suspending agent, a preservative, or the like.

In one aspect, the composition according to the invention may be prepared with an aqueous solution for non-oral administration, and preferably, Hank's solution, Ringer's solution, or a buffer solution such as physically buffered saline may be used. A water-soluble injection suspension may include a substrate capable of increasing the viscosity of a suspension such as sodium carboxymethyl cellulose, sorbitol, or dextran. The composition of the present invention may be systemically or locally administered and may be formulated into an appropriate dosage form according to known techniques for such administration. For example, for oral administration, the composition may be mixed with an inert diluent or an edible carrier, sealed in a hard or soft gelatin capsule, or formulated as a tablet. In the case of oral administration, the active compound may be mixed with an excipient and used in the form of ingestible tablets, buccal tablets, troches, capsules, elixirs, suspensions, syrups, wafers, or the like. Various dosage forms for injection, parenteral administration, and the like may be prepared based on techniques known in the art or commonly practiced methods. For administration of a dosage form, intravenous injection, subcutaneous injection, intramuscular injection, peritoneal injection, percutaneous administration, and the like may be used. The appropriate dosage of the composition of the present invention may be variously prescribed depending on such factors as formulation method, administration method, the age, weight, gender, and morbidity of a patient, food, administration time, administration route, excretion rate, and reaction sensitivity. For example, the composition of the present invention may be administered to adults at a dosage of 0.1 to 1,000 mg/kg, preferably at a dosage of 10 to 100 mg/kg, once or several times daily.

1. A method of enhancing therapeutic activity of a T regulatory cells comprising enhancing mobility of said cell towards a chemotactic gradient.

2. The method of claim 1, wherein said T regulatory cell is derived from a pluripotent stem cell.

3. The method of claim 2, wherein said pluripotent stem cell is an inducible pluripotent stem cell.

4. The method of claim 3, wherein said inducible pluripotent stem cell is generated through transfection of dedifferentiation factors into a somatic cell.

5. The method of claim 4, wherein said somatic cell is a mesenchymal stem cell.

6. The method of claim 5, wherein said tissue derived mesenchymal stem cell is isolated from umbilical cord tissue.

7. The method of claim 1, wherein said T regulatory cell possesses enhanced cytoskeleton mobility properties as compared to a nonmanipulated T regulatory cell.

8. The method of claim 7, wherein said cytoskeleton is modified by enhancing expression of vimentin in T regulatory cells.

9. The method of claim 8, wherein said expression of vimentin is enhanced by positioning the vimentin gene under control of the FoxP3 promoter.

10. The method of claim 9, wherein said vimentin gene is additionally placed under control of HIF-1 alpha inducible promoter.

11. The method of claim 1, wherein said therapeutic activity of said T regulatory cell is stimulation of endogenous progenitor cells.

12. The method of claim 11, wherein said endogenous progenitor cells are cardiac stem specific stem cells.

13. The method of claim 12, wherein said cardiac specific stem cells express c-kit.

14. The method of claim 11, wherein said endogenous progenitor cells are osteoblastic cells.

15. The method of claim 14, wherein said osteoblastic cells are capable of generating bone cells.

16. The method of claim 15, wherein said osteoblastic cells are inhibited by RANK ligand.

17. The method of claim 16, wherein said endogenous progenitor cells are chondrocyte progenitor cells.

18. The method of claim 15, wherein said endogenous progenitor cells are pancreatic progenitors.

19. The method of claim 1, wherein said enhanced migration of said T regulatory cell is provided for by treatment of said T regulatory cell with a dedifferentiation agent or a combination of a dedifferentiation agent and a chemotaxis promoting agent.

20. The method of claim 19, wherein said dedifferentiation agent is a histone deacetylase inhibitor.
